(19) United States
(12) Patent Application Publication
(10) Pub. No.: US 2026/0360507 A1
(43) Pub. Date: Nov. 12, 2026

(54) PERSONALIZED HEALTH MANAGEMENT USING DIGITAL TWINS

(71) Applicant: Medtronic MiniMed, Inc., Northridge, CA (US)

(72) Inventors: Andrew Thomas Fried, Saint Paul, MN (US); Alfonso Santiago Ferrandez, Minneapolis, MN (US); Markus W. Reiterer, Plymouth, MN (US); Louis J. Lintereur, Boise, ID (US); Cahal McVeigh, White Bear Township, MN (US); Gwendolyn K. Davis, Minneapolis, MN (US); Kevin Michael Kelleher, Troy, OH (US)

(21) Appl. No.: 19/667,890

(22) Filed: May 5, 2026

Related U.S. Application Data
(60) Provisional application No. 63/801,263, filed on May 7, 2025.
(60) Provisional application No. 63/814,095, filed on May 29, 2025.

(51) Int. Cl. G16H 20/60; G06F 3/16; G06T 7/70; G06V 10/70; G16H 20/10; G16H 40/67; G16H 50/30

(52) U.S. Cl. CPC G16H 20/60; G06F 3/16; G06T 7/70; G06V 10/70; G16H 20/10; G16H 40/67; G16H 50/30; G06T 2207/20081; G06T 2207/30196

(57) ABSTRACT

A system may include one or more processors and processor-readable media storing instructions that, when executed by the one or more processors, may cause the system to obtain behavioral data and biometric data relating to an individual, maintain a digital twin of the individual including a physiological model of the individual, maintain a patient belief profile representing health-related beliefs of the individual, dynamically update the digital twin based on new information obtained about the individual, and deliver one or more personalized health interventions based on the behavioral data, the biometric data, the digital twin, the patient belief profile, or a combination thereof. In some embodiments, the system may interface with a human health knowledge model, identify a knowledge gap or false belief, simulate predicted physiological outcomes, generate rewards, generate meal plans, communicate aspects of the digital twin to a clinical user, and incorporate outputs from a home-based monitoring subsystem.

### RELATED APPLICATIONS

This application claims the benefit of U.S. Provisional Application No. 63/801,263, filed May 7, 2025, entitled “SYSTEMS AND METHODS FOR CHANGING HUMAN BEHAVIOR AND LIFESTYLE CHOICES FOR REVERSING AND PREVENTING DISEASE,” and claims the benefit of U.S. Provisional Application No. 63/814,095, filed May 29, 2025, entitled “SYSTEMS AND METHODS FOR REMOTE MONITORING AND MANAGEMENT OF DIABETES USING AI-POWERED INVISIBLE AND MULTIMODAL SENSING TECHNOLOGIES,” which is assigned to the assignee hereof, and incorporated herein in its entirety by reference.

### FIELD

The disclosure relates to the healthcare and wellness industries and the ability of systems to influence the behavior of individuals and households.

### BACKGROUND

Developed nations, in particular the United States of America, are facing a healthcare and wellness crisis. The United States healthcare system has not adequately prepared society for how to make healthy lifestyle choices, such as how to eat. The U.S. healthcare system is currently using reductionist practices, treating symptoms of parts of the body, not the whole person, but most early deaths in the U.S. are preventable through diet or other behavioral choices. This results in lower life expectancies and higher costs of healthcare in the U.S. compared to other countries. The reality is that more than one-third of U.S. adults are obese, increasing risk for heart disease, stroke, type 2 diabetes, and some cancers. Certain demographics in certain parts of the U.S. have a one in two chance of obesity. Type 2 diabetes affects a significant portion of Americans and drives disability and early death. Suicide rates have been on the rise and are a top cause of death for teenagers and young adults. U.S. homicide rates are ~7 times higher than in peer nations. The lifetime cancer risk in the U.S. is ~39.5% (1 in 2.5 people). U.S. county of birth accounts for up to 27 years of life expectancy. The good news is that many of these afflictions are preventable and reversible through lifestyle choices.

### SUMMARY

In general, this disclosure is directed to personalized health management that combines continuous monitoring, individualized coaching, and clinical decision support. A processing system may maintain a digital twin that models an individual’s physiological systems, and a separate patient belief profile that captures the individual’s health-related beliefs. By comparing the patient’s belief profile against a curated knowledge base of human health, the system may identify knowledge gaps or false beliefs and deliver targeted educational content. The system may simulate the digital twin to predict how behaviors or actions may affect the individual’s health, and may provide rewards in association with healthy behaviors. In some embodiments, a home-based monitoring subsystem may use cameras and local processing to generate a timeline of the individual’s activities and inferred physiological parameters, transmitting only derived outputs to the digital twin while deleting raw image data locally. The system may deliver personalized health interventions based on the digital twin, and may provide healthcare providers with an interface for reviewing patient data and delivering care.

An example system, according to this description, includes one or more processors and one or more processor-readable media storing instructions which, when executed by the one or more processors, cause performance of operations comprising obtaining behavioral data relating to an individual, obtaining biometric data relating to the individual, maintaining a digital twin of the individual comprising a physiological model of the individual, maintaining a patient belief profile, wherein the patient belief profile represents health-related beliefs of the individual, dynamically updating the digital twin based on new information obtained about the individual, and delivering one or more personalized health interventions to the individual based on the behavioral data, the biometric data, the digital twin, the patient belief profile, or a combination thereof.

An example method, according to this description, includes obtaining, by a processing system, behavioral data relating to an individual, obtaining, by the processing system, biometric data relating to the individual, maintaining, by the processing system, a digital twin of the individual comprising a physiological model of the individual, maintaining, by the processing system, a patient belief profile, wherein the patient belief profile represents health-related beliefs of the individual, dynamically updating, by the processing system, the digital twin based on new information obtained about the individual, and delivering, by the processing system, one or more personalized health interventions to the individual based on the behavioral data, the biometric data, the digital twin, the patient belief profile, or a combination thereof.

An example non-transitory computer-readable medium, according to this description, stores instructions that, when executed by a processing system, cause the processing system to obtain behavioral data relating to an individual, obtain biometric data relating to the individual, maintain a digital twin of the individual comprising a physiological model of the individual, maintain a patient belief profile, wherein the patient belief profile represents health-related beliefs of the individual, dynamically update the digital twin based on new information obtained about the individual, and deliver one or more personalized health interventions to the individual based on the behavioral data, the biometric data, the digital twin, the patient belief profile, or a combination thereof.

In some embodiments, image or sensor data may be obtained from a dedicated home-based monitoring subsystem or from a user device. Thermal image processing may support monitoring during sleep or other low-motion periods and may contribute to respiratory or metabolic inference. Machine learning operations may be implemented using a staged model architecture, a single end-to-end model, or multiple specialized models. A physiological inference model may be trained using synthetic data generated from a physiological body model, additionally, or alternatively, using measured data. Alert routing may be tiered among the individual, a caregiver, a healthcare provider, and an emergency endpoint based on severity and responsiveness. The system may also aggregate pharmacy, laboratory, wearable-platform, and electronic health record data into a longitudinal health-event record associated with the digital twin.

In some embodiments, the system may provide multiple patient-facing interfaces, including a mobile application, a smart display, or a smart mirror configured to present health summaries, reminders, coaching, and alerts. The patient belief profile may be updated longitudinally using timestamped evidence derived from interactions, behaviors, and conversational data, and portions of the patient belief profile may represent uncertainty where evidence is unavailable. The curated knowledge base may encode context-dependent health relationships, and a comparison engine may identify partial inconsistencies, uncertainty, or condition-specific knowledge gaps. A clinician interface may present summarized patient status, drill-down views, trends, population comparisons, and predicted future states derived from the digital twin.

Additional features and embodiments are described in the accompanying drawings and the description below.

### BRIEF DESCRIPTION OF DRAWINGS

The above and other aspects and features of the disclosure will become more apparent in view of the following detailed description when taken in conjunction with the accompanying drawings wherein like reference numerals identify like elements.

FIG. 1 illustrates an overall system architecture for personalized health management according to some embodiments.

FIG. 2 illustrates an application architecture for a user-facing application according to some embodiments.

FIG. 3A illustrates a digital twin structure according to some embodiments.

FIG. 3B illustrates a patient belief profile structure according to some embodiments.

FIG. 4 illustrates a human health knowledge model structure according to some embodiments.

FIG. 5 illustrates a gamification state machine according to some embodiments.

FIG. 6 illustrates a message flow diagram for identifying knowledge gaps or false beliefs and delivering personalized coaching and education according to some embodiments.

FIG. 7A illustrates a meal planning workflow according to some embodiments.

FIG. 7B illustrates a food logging and digital twin simulation workflow according to some embodiments.

FIG. 8 illustrates a clinician interface system according to some embodiments.

FIG. 9 illustrates a home-based remote monitoring system architecture according to some embodiments.

FIG. 10 illustrates an edge computing device internal processing architecture according to some embodiments.

FIG. 11 illustrates an integration architecture for combining the home-based remote monitoring subsystem with the user-facing application via a shared digital twin according to some embodiments.

FIG. 12 illustrates a computer system according to some embodiments.

FIG. 13 illustrates a method for personalized health management according to some embodiments.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the present disclosure. Other embodiments may be utilized, and structural, logical, and electrical changes may be made without departing from the scope of the disclosure. The various embodiments are not necessarily mutually exclusive, as some embodiments can be combined with one or more other embodiments to form new embodiments.

As used herein, the term “digital twin” refers to a computational model that represents physiological systems of an individual. The digital twin may comprise a hierarchical model of human body systems, including circulatory, respiratory, digestive, immune, nervous, endocrine, and musculoskeletal systems, among others. The physiological systems may be modeled using one or more types of models, such as mathematical models, machine learning models, artificial intelligence models, statistical models, or hybrid models combining multiple modeling approaches. The digital twin may be dynamically updated based on behavioral data, biometric data, and other information received about the individual.

As used herein, the term “patient belief profile” refers to a data structure associated with a digital twin that represents health-related beliefs of an individual. The patient belief profile may be derived from interactions with the individual, behavioral observations, and other inputs. For example, the patient belief profile may be constructed by analyzing conversational data from interactions with a virtual health coach, where statements such as “I believe skipping breakfast helps me lose weight” or “I think my medication causes more harm than good” or “red meat is good for me, it’s all I eat” or “I love sweets like cookies and having a few a day is fine” or “Cola is my favorite beverage when I watch TV in the evening” or “I eat a cereal with milk every morning” are extracted and stored. The patient belief profile may be implemented as a structured knowledge graph, a relational database of belief statements with associated confidence scores, or a machine learning model trained on the individual’s language and behavior patterns. The patient belief profile may be compared against a curated knowledge base to identify knowledge gaps or false beliefs. For example, if the patient belief profile indicates that the individual believes a particular food is healthy, but the curated knowledge base indicates that the food is associated with adverse or sub-optimal health outcomes for individuals with the patient’s health conditions or for people in general, the system may identify this as a false belief and deliver targeted educational content.

As used herein, the term “edge computing device” refers to a computing device that processes data locally, typically within or near the environment where data is generated, rather than transmitting raw data to a remote server or cloud system for processing. In the context of a home-based monitoring subsystem, an edge computing device may comprise a dedicated hardware appliance installed in the home, a general-purpose computer configured with monitoring software, or a smart home hub with integrated processing capabilities. The edge computing device may receive video feeds from one or more cameras, process the video frames using machine learning models to detect poses and infer physiological parameters, and generate derived outputs such as activity timelines and risk scores. The edge computing device may delete raw image data after processing to preserve privacy, transmitting only non-image-derived parameters to remote systems.

As used herein, the term “activity timeline” refers to a time-series representation of activity states and inferred physiological parameters of an individual over one or more time periods. The activity timeline may comprise variable time-step windows that adapt based on detected activity levels. For example, during periods of sleep or inactivity, the activity timeline may use longer time windows (e.g., 10-minute intervals), while during periods of active movement or detected anomalies, the activity timeline may use shorter time windows (e.g., 1-minute or sub-minute intervals) to capture rapid changes in state. Each entry in the activity timeline may include an activity label (e.g., sleeping, walking, cooking, sitting, standing), one or more inferred physiological parameters (e.g., estimated heart rate, respiratory rate, body temperature), and a timestamp. The activity timeline may be stored locally on an edge computing device or in a user-facing application and transmitted periodically or in response to detected events to a remote processing system for incorporation into the digital twin.

As used herein, the terms “individual” and “user” refer to a person who uses the system or for whom data is collected, processed, or analyzed. This encompasses users of the user-facing application, patients under clinical care, participants in wellness programs, or any other person whose health is managed or monitored by the system. The terms “user,” “patient,” and “participant” may be used interchangeably with “individual” in certain contexts.

Health management systems may collect data from multiple sources, but may not integrate that data into a unified, personalized model of an individual's health. Individuals may hold beliefs about health that are inconsistent with scientific knowledge, and conventional systems may not identify or address these knowledge gaps. Remote monitoring systems may rely on wearable sensors that require continuous individual compliance, and camera-based approaches may raise privacy concerns if raw image data is transmitted outside the home environment. Healthcare providers may lack tools for synthesizing diverse data sources into a coherent view that supports clinical decision-making and individual engagement.

Disclosed herein are systems and methods that address these challenges by maintaining a digital twin of an individual comprising a physiological model, and a patient belief profile associated with the digital twin. A processing system may compare the patient belief profile against a human health knowledge model to identify knowledge gaps or false beliefs, and may deliver educational content to the individual based on identified gaps. The processing system may simulate the digital twin to predict physiological outcomes based on behaviors or actions, and may output the predicted outcomes to the individual. In some embodiments, a smart home health system comprising a monitoring subsystem may capture image data of the individual within a home environment and process the image data locally on an edge computing device to generate an activity timeline. The edge computing device may delete raw image data after processing, such that no raw image data is transmitted outside the home environment. Derived outputs from the activity timeline may be transmitted for incorporation into the digital twin. The system may deliver personalized health interventions based on the digital twin, and may provide a clinician interface for healthcare providers to review and act on patient data.

The disclosed systems and methods may provide several advantages. By maintaining a separate patient belief profile and comparing it against a curated knowledge base, the system may identify and address knowledge gaps or false beliefs that influence health outcomes. By simulating the digital twin, the system may provide individualized predictions of physiological responses to behaviors or actions, enabling more informed decision-making by the individual. By processing image data locally on an edge computing device and deleting raw image data after processing, the system may preserve privacy while enabling continuous monitoring of activity and physiological state. By integrating data from multiple sources into a unified digital twin, the system may provide healthcare providers with a coherent, actionable view of patient health. By delivering personalized health interventions based on the digital twin, the system may support continuous engagement and behavior change between clinical encounters.

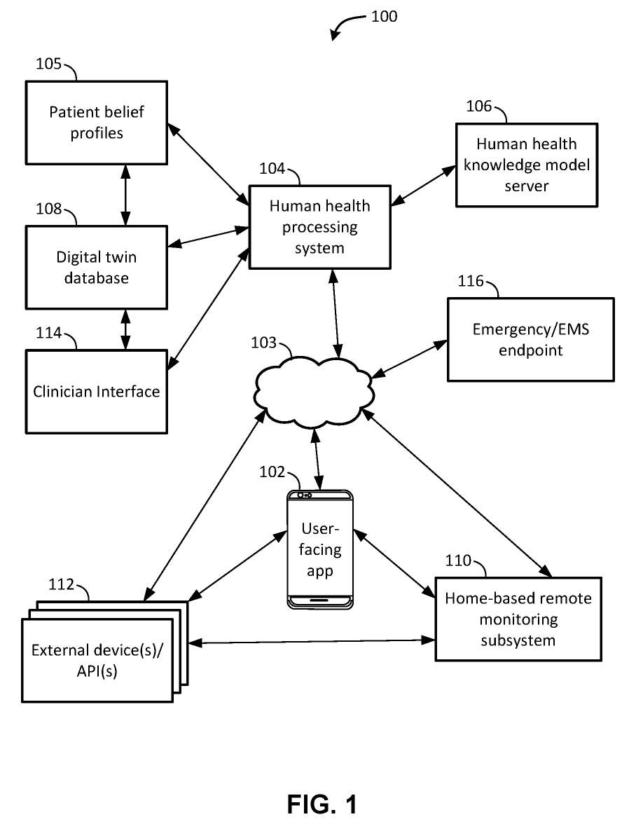

FIG. 1 is a block diagram illustrating an overall system architecture 100 for personalized health management according to some embodiments. The system architecture 100 may be provided as an example, and embodiments may vary in configuration, component selection, and interconnection topology. (E.g., arrows representing communication channels may vary, depending on desired functionality.) The system architecture 100 may comprise a user-facing application 102 that provides an interface for an individual. The user-facing application 102 may be implemented as a mobile application executing on a smartphone, tablet, or wearable device, a web application accessible via a browser, or a desktop application. The user-facing application 102 may communicate with a human health processing system 104 via a network 103. The network 103 may comprise the Internet, a cellular network, a local area network, a wide area network, or any combination thereof. The human health processing system 104 may comprise one or more servers, cloud-based computing resources, distributed computing nodes, or edge computing devices configured to perform data processing, model simulation, and intervention delivery functions. The human health processing system 104 may be implemented using virtual machines, containerized services, serverless computing functions, or dedicated hardware appliances.

The system architecture 100 may further comprise patient belief profiles 105 associated with the digital twins stored in the digital twin database 108. Each patient belief profile 105 may represent health-related beliefs of a corresponding individual and may be stored as a structured data file, a knowledge graph, a relational database, a series of session files, or a machine learning model. The patient belief profiles 105 may be maintained by the human health processing system 104 and updated based on interactions with individuals via the user-facing application 102.

The system architecture 100 may further comprise a human health knowledge model server 106 that maintains a curated knowledge base of human physiology and interactions with diet, exercise, pharmacological agents, and environmental factors. The human health knowledge model server 106 may be implemented as a database server, a knowledge graph server, or a cloud-based data service. The human health processing system 104 may interface with the human health knowledge model server 106 via the network 103 to perform comparisons between patient belief profiles and the curated knowledge base.

The system architecture 100 may further comprise a digital twin database 108 that stores digital twins for a plurality of individuals. The digital twin database 108 may be implemented as a relational database, a time-series database, or a distributed data store, for example. Each digital twin may comprise a physiological model of an individual and may be associated with a corresponding patient belief profile. In some cases, the digital twin database 108 parameters for an individual may be changed as a result of the patient belief profile 105. For example, if a patient believes that large carbohydrate intake right before bed improves sleep, the digital twin for that patient may include a feeding session at a particular time in the evening with a particular probability. The digital twin database 108 may be accessible by the human health processing system 104 for reading and updating digital twin data via the network 103.

The digital twin database 108 may additionally, or alternatively, store a longitudinal health-event record for the individual. The longitudinal health-event record may aggregate information from the user-facing application 102, the home-based remote monitoring subsystem 110, external devices and APIs 112, clinician-entered data, laboratory systems, pharmacy systems, and other sources over extended periods of time. The longitudinal health-event record may include timestamps for symptoms, meals, medication events, exercise events, sleep intervals, physiological measurements, alerts, clinical encounters, diagnoses, procedures, and interventions. The processing system may use the longitudinal health-event record to identify causal patterns, personalize simulations, generate clinician summaries, or refine baseline models for anomaly detection.

The system architecture 100 may further comprise external devices and APIs 112, which may include wearable devices, biosensors, smart scales, continuous glucose monitors, fitness trackers, third-party health applications, implantable medical devices, and other data sources. The external devices and APIs 112 may communicate with the user-facing application 102 or directly with the human health processing system 104 via the network 103 to provide behavioral data and biometric data.

The system architecture 100 may further comprise a home-based remote monitoring subsystem 110 that includes one or more cameras and an edge computing device configured to capture and process image data locally. The remote monitoring subsystem 110 may communicate with the human health processing system 104 and the digital twin database 108 via the network 103. The remote monitoring subsystem 110 may transmit derived outputs to the digital twin database 108 for incorporation into the digital twin, while deleting raw image data locally to preserve privacy.

The system architecture 100 may further comprise a clinician interface 114 that enables healthcare providers to review patient data, access digital twin information, and deliver care. The clinician interface 114 may be implemented as a web-based portal, a desktop application, or a mobile application. The clinician interface 114 may communicate with the human health processing system 104 and the digital twin database 108 via the network 103 to retrieve patient information and provide clinical input. Depending on desired functionality, this interaction may involve direct or indirect information exchanges with other entities of the system architecture 100, as needed to retrieve the patient information and/or provide the clinical input. Such entities may include, for example, the patient belief profiles 105 and the human health knowledge model server 106.

The system architecture 100 may further comprise an emergency services endpoint 116 that may receive escalated alerts from the remote monitoring subsystem 110 via the network 103 in the event of detected anomalies that require immediate medical attention. The emergency services endpoint 116 may comprise an emergency medical services dispatch system, a monitoring center, or an automated alert relay service.

In operation, the system architecture 100 may enable continuous monitoring and personalized health management for individuals. The user-facing application 102 may collect behavioral data and biometric data from the individual and from external devices and APIs 112, and may transmit this data to the human health processing system 104 via the network 103. The human health processing system 104 may update the digital twin stored in the digital twin database 108 based on the received data. The home-based remote monitoring subsystem 110 may capture image data of the individual, process the image data locally to generate activity timelines and inferred physiological parameters, and transmit derived outputs to the digital twin database 108 for incorporation into the digital twin. The human health processing system 104 may compare the patient belief profile 105 associated with the individual’s digital twin against the human health knowledge model maintained by the human health knowledge model server 106 to identify knowledge gaps or false beliefs. Based on identified knowledge gaps, the current state of the digital twin, and detected activity patterns, the human health processing system 104 may deliver personalized health interventions to the individual via the user-facing application 102. These interventions may include educational content, predicted physiological outcomes based on simulation of the digital twin, personalized audio outputs such as guided meditation, meal plans, and rewards for healthy behaviors. The clinician interface 114 may enable healthcare providers to review the digital twin and patient belief profiles, assess trends and anomalies, and provide clinical guidance that is delivered to the individual via the user-facing application 102. In the event of detected anomalies that exceed risk thresholds, the remote monitoring subsystem 110 may transmit alerts to the individual via the user-facing application 102, to healthcare providers via the clinician interface 114, or to the emergency services endpoint 116 if immediate medical attention is required.

The system architecture 100 may further support population-level analysis and refinement of the human health knowledge model by aggregating data from digital twins stored in the digital twin database 108. The human health processing system 104 may analyze de-identified or anonymized data from a plurality of digital twins to identify trends, correlations, and patterns across populations, geographies, comorbidities, or other factors. For example, the human health processing system 104 may identify behavioral patterns associated with particular health outcomes, such as meal timing strategies, exercise routines, or sleep hygiene practices that correlate with glycemic variability or cardiovascular health. The human health processing system 104 may identify treatment or therapy patterns by comparing outcomes across individuals with similar health conditions, comorbidities, or demographic characteristics. The human health processing system 104 may update the human health knowledge model maintained by the human health knowledge model server 106 based on population-level insights and over time; in this way the human health knowledge model may continue to improve and gain knowledge that traditional scientific methods may not. The human health processing system 104 may also refine machine learning models used in digital twin simulation or physiological parameter inference based on aggregated population data over time.

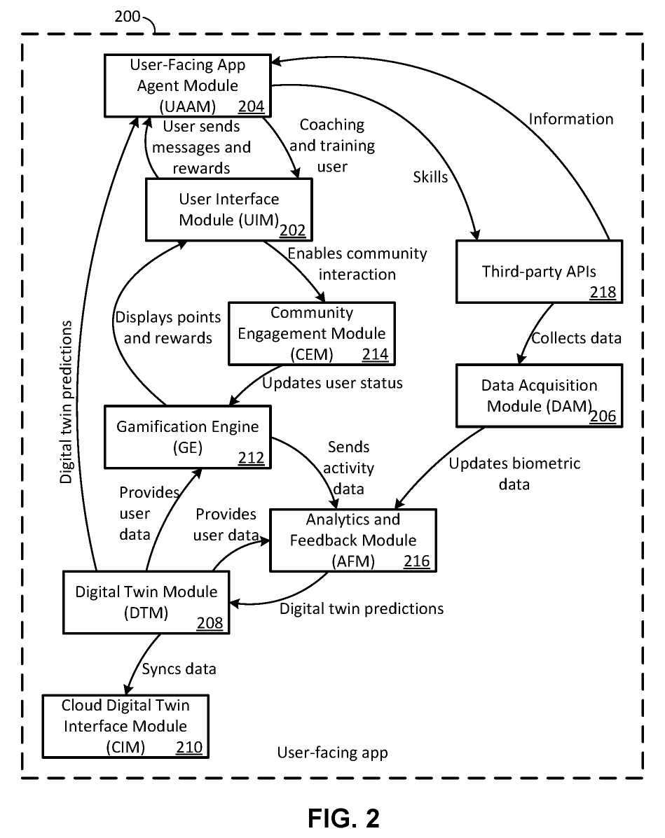

FIG. 2 illustrates an application architecture 200 for a user-facing application according to some embodiments. The application architecture 200 may be provided as an example, and embodiments may vary in module configuration, interconnection, and functionality. Interconnections shown are example embodiments, but in practice other interconnections may be used between sub-modules. In this example, unidirectional connections are shown, but in practice bidirectional connections may be appropriate. The application architecture 200 may represent the internal structure of the user-facing application 102 shown in FIG. 1.

The application architecture 200 may comprise a user interface module 202 that provides visual and interactive elements for the individual to interact with the application. The user interface module 202 may be implemented using native mobile application frameworks, web-based user interface technologies, or cross-platform development frameworks. The user interface module 202 may present information to the individual, receive user input, and coordinate the display of content from other modules.

The application architecture 200 may further comprise a user-facing app agent module 204 that provides personalized coaching and conversational interaction with the individual. The user-facing app agent module 204 may be implemented using natural language processing models, generative AI systems, chatbot frameworks, or rule-based dialogue systems. The user-facing app agent module 204 may deliver health interventions, educational content, and motivational messaging based on the individual’s digital twin and patient belief profile.

In some embodiments, the user-facing app agent module 204 may generate personalized therapeutic audio outputs based on parameters of the digital twin. The therapeutic audio outputs may include guided meditation sessions, hypnotic suggestions, binaural beats, alpha wave audio, or other audio content designed to support mental health, stress reduction, sleep quality, or behavior change. The personalized therapeutic audio may be tailored based on the individual’s current physiological state, stress levels, sleep patterns, psychological profile, or health goals as reflected in the digital twin. For example, if the digital twin indicates elevated cortisol levels or poor sleep quality, the user-facing app agent module 204 may generate a guided meditation session with calming narration and background music designed to reduce stress and promote relaxation. The therapeutic audio may incorporate binaural beats, which are auditory processing artifacts created when two tones of slightly different frequencies are presented separately to each ear, potentially influencing brainwave patterns and promoting states such as relaxation, focus, or sleep. The therapeutic audio may incorporate alpha wave audio, which may include tones or music designed to encourage alpha brainwave activity associated with relaxed wakefulness and reduced anxiety. The user-facing app agent module 204 may deliver hypnotic suggestions in the form of guided audio scripts that use relaxation techniques, focused attention, and suggestive language to support behavior change, reduce cravings, improve sleep, or address other health-related goals. The therapeutic audio outputs may be delivered via the user interface module 202, through speakers of the device executing the application, or through connected audio devices such as headphones, wireless speakers, or smart speakers.

The application architecture 200 may further comprise a data acquisition module 206 that collects data, such as behavioral data, pharmacological data, and biometric data or other data from various sources. The data acquisition module 206 may interface with sensors on the device executing the application, such as accelerometers, gyroscopes, or GPS receivers. The data acquisition module 206 may also interface with external devices and APIs to retrieve data from wearable devices, biosensors, smart scales, and third-party health applications.

The application architecture 200 may further comprise a digital twin module 208 that maintains a local copy of the individual’s digital twin. The digital twin module 208 may store a subset of the digital twin data locally on the device for offline access and rapid retrieval. The digital twin module 208 may synchronize with the digital twin database 108 shown in FIG. 1 via a cloud digital twin interface module 210.

The application architecture 200 may further comprise the cloud digital twin interface module 210 that manages communication between the local digital twin module 208 and the remote digital twin database 108. The cloud digital twin interface module 210 may transmit updates to the digital twin based on newly acquired data, and may retrieve updated digital twin data from the remote database when network connectivity is available.

The application architecture 200 may further comprise a gamification engine 212 that implements reward mechanisms to encourage healthy behaviors. The gamification engine 212 may track activities performed by the individual, calculate rewards based on predefined criteria or dynamic goals, and present rewards to the individual via the user interface module 202. The gamification engine 212 may support individual rewards and group rewards for behaviors performed by multiple individuals.

The application architecture 200 may further comprise a community engagement module 214 that enables social interaction among individuals using the application. The community engagement module 214 may provide a social feed for sharing content, posting about healthy activities, and viewing community events. The community engagement module 214 may implement content moderation to identify and flag false or misleading health information.

The application architecture 200 may further comprise an analytics and feedback module 216 that processes data collected by the data acquisition module 206 and generates insights for presentation to the individual. The analytics and feedback module 216 may calculate trends, identify patterns, and generate visualizations of health metrics over time. The analytics and feedback module 216 may provide input to the user-facing app agent module 204 to inform personalized coaching.

The application architecture 200 may further comprise third-party APIs 218 that enable integration with external services. The third-party APIs 218 may include APIs for grocery ordering, meal delivery, fitness class booking, telehealth services, or other health-related services. The user-facing app agent module 204 or other modules may invoke the third-party APIs 218 to perform actions on behalf of the individual.

The third-party APIs 218 may additionally, or alternatively, include interfaces to pharmacy systems, prescription management systems, laboratory information systems, electronic health record systems, provider scheduling systems, payer systems, wearable-device platforms, nutrition databases, and retail ordering systems. Through such interfaces, the system may obtain medication fill status, refill history, laboratory results, diagnoses, care plans, appointment information, device-generated physiological data, or other information relevant to the digital twin. The system may also transmit outputs such as refill requests, appointment requests, telehealth requests, care-coordination messages, or structured summaries generated from the digital twin.

In some embodiments, the third-party APIs 218 may include interfaces to neurofeedback systems, biofeedback devices, meditation platforms, mental health applications, or therapeutic audio services. For example, the third-party APIs 218 may interface with commercially available neurofeedback systems that provide real-time brainwave monitoring and feedback to support meditation practice, stress reduction, or sleep improvement. Such neurofeedback systems may measure electroencephalography (EEG) signals via a wearable headband or other sensor configuration and may provide audio feedback to guide the individual toward desired mental states. The system may retrieve neurofeedback data from the neurofeedback system API, including brainwave activity patterns, meditation session duration, calmness scores, or sleep stage information, and may incorporate this data into the digital twin. The system may also interface with third-party APIs for Eye Movement Desensitization and Reprocessing (EMDR) therapy applications, which may provide guided EMDR sessions to support treatment of trauma, anxiety, or other psychological conditions. The system may interface with third-party APIs for hypnosis applications, guided meditation platforms, binaural beat generators, or other therapeutic audio services. The user-facing app agent module 204 may invoke these third-party APIs to deliver therapeutic interventions to the individual, and may receive feedback data from these services for incorporation into the digital twin and patient belief profile.

In operation, the modules of the application architecture 200 may work together to provide a health management workflow. The data acquisition module 206 may continuously or periodically collect data from sensors and external devices. The analytics and feedback module 216 may process the collected data and update the digital twin module 208. The cloud digital twin interface module 210 may synchronize the local digital twin with the remote digital twin database 108 when connectivity is available. The user-facing app agent module 204 may retrieve information from the digital twin module 208 and deliver personalized interventions via the user interface module 202. The gamification engine 212 may award rewards based on detected healthy behaviors, and the community engagement module 214 may enable the individual to share achievements and participate in group activities. The user-facing application 102 may interface with the human health processing system 104 to access the patient belief profiles 105 and the human health knowledge model server 106 for knowledge gap identification and educational content generation. The user-facing application 102 may also interface with other the elements of system architecture 100 not shown in FIG. 2, such as the home-based remote monitoring subsystem 110, the external devices and APIs 112, the clinician interface 114, the emergency services endpoint 116, and the like.

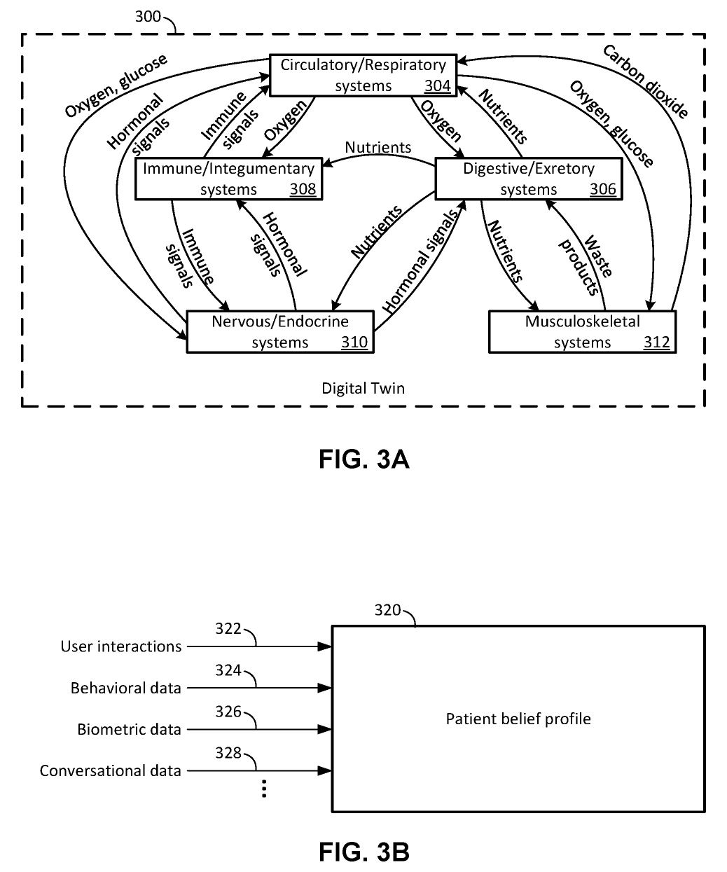

FIG. 3A is a diagram illustrating a digital twin structure 300 according to some embodiments. The digital twin structure 300 may be provided as an example, and embodiments may vary in organization, level of detail, and modeling approach. The digital twin structure 300 may represent the structure of a digital twin stored in the digital twin database 108 shown in FIG. 1, maintained locally by the digital twin module 208 shown in FIG. 2, or shared across both.

The digital twin structure 300 may comprise a hierarchical physiological model that decomposes the human body into a plurality of interconnected systems, each representing a major physiological system. The hierarchical physiological model may include a circulatory and respiratory system 304, a digestive and excretory system 306, an immune and integumentary system 308, a nervous and endocrine system 310, and a musculoskeletal system 312. Each system may be further decomposed into subsystems, organs, tissues, or functional units as appropriate for the level of modeling detail required.

The circulatory and respiratory system 304 may model cardiovascular function, blood flow, oxygen transport, respiratory mechanics, and gas exchange. The circulatory and respiratory system 304 may include parameters such as heart rate, blood pressure, cardiac output, respiratory rate, oxygen saturation, and lung capacity. The digestive and excretory system 306 may model nutrient absorption, metabolism, waste elimination, and fluid balance. The digestive and excretory system 306 may include parameters such as blood glucose levels, ketone levels, insulin sensitivity, metabolic rate, hydration level/status, and kidney function. The immune and integumentary system 308 may model immune response, inflammation, skin health, and barrier function. The immune and integumentary system 308 may include parameters such as white blood cell counts, inflammatory markers, skin temperature, and wound healing status. The nervous and endocrine system 310 may model neural signaling, hormonal regulation, stress response, and cognitive function. The nervous and endocrine system 310 may include parameters such as insulin levels, cortisol levels, thyroid hormone levels, antidiuretic hormone levels, testosterone levels, estrogen levels, neurotransmitter activity, and sleep quality metrics. The musculoskeletal system 312 may model muscle strength, bone density, joint mobility, and physical performance. The musculoskeletal system 312 may include parameters such as muscle mass, bone mineral density, range of motion, and exercise capacity.

The allocation of specific parameters to particular physiological systems in the digital twin structure 300 is illustrative and may vary between implementations. Some physiological parameters may be relevant to multiple systems and may be shared across system boundaries. For example, blood glucose levels, insulin levels, hydration status, and ketone levels may be monitored and utilized by both the circulatory and respiratory system 304 and the digestive and excretory system 306, as well as other systems. In some embodiments, a parameter may be stored in association with one system but accessed or referenced by multiple systems through the interconnections shown in FIG. 3A. In other embodiments, a parameter may be duplicated across multiple systems, with synchronization mechanisms ensuring consistency. The specific organizational structure of parameters within the digital twin may be adapted based on modeling requirements, computational efficiency, clinical conventions, or other implementation considerations.

The physiological systems may be interconnected, representing physiological interactions, feedback loops, and parameter dependencies between systems. For example, the circulatory and respiratory system 304 may provide oxygen to the musculoskeletal system 312, and the musculoskeletal system 312 may influence cardiovascular demand during physical activity. The digestive and excretory system 306 may provide glucose to multiple systems, and the nervous and endocrine system 310 may regulate insulin secretion in response to blood glucose levels. The labels in FIG. 3A show examples of information that may be shared by these interconnections. These interconnections may enable simulation of the digital twin to predict how changes in one system affect other systems and overall physiological state.

Each physiological system may have associated parameters that are dynamically updated based on incoming behavioral data, biometric data, sensor measurements, and clinical observations. The parameters may be used as inputs for the simulation of the digital twin to generate predicted physiological outcomes. The digital twin may be calibrated or personalized over time as new data becomes available, improving the accuracy of the model for the individual.

The digital twin structure 300, including the various systems shown and subsystems thereof, may be implemented using one or more modeling approaches. Mathematical models such as differential equations, lumped-parameter models, or compartmental models may represent physiological dynamics. Machine learning models such as neural networks, decision trees, or ensemble models may be trained on population data or individual historical data to predict physiological responses. Hybrid models may combine mathematical and machine learning approaches to leverage the strengths of each. The digital twin structure 300 may be updated dynamically as new data is received, and may be calibrated or personalized based on the individual’s unique physiological characteristics and responses. As knowledge of the human body grows and the human health knowledge model server 106 improves, the digital twin structure 300 may also change according to new information.

FIG. 3B illustrates a patient belief profile structure 320 according to some embodiments. The patient belief profile structure 320 may be provided as an example, and embodiments may vary in the types of input data used and the manner in which beliefs are represented. The patient belief profile structure 320 may represent a patient belief profile 105 shown in FIG. 1, and may be associated with a digital twin, such as the digital twin structure 300 shown in FIG. 3A.

The patient belief profile 320 may be constructed from multiple types of input data. User interactions 322 may include responses to prompts, selections made within the user-facing application 102, engagement with educational content, and other direct interactions with the application interface. Behavioral data 324 may include observed behaviors such as meal choices, exercise patterns, sleep habits, medication adherence, and other actions that may reflect underlying beliefs about health. Biometric data 326 may include physiological measurements from sensors or wearable devices that may correlate with health-related beliefs or practices. Conversational data 328 may include text, audio, or video exchanges between the individual and the user-facing app agent module 204, from which statements about health beliefs may be extracted. Other input types (not shown) may include social media activity, responses to surveys or questionnaires, interactions with community features, information from the home-based remote monitoring subsystem 110, information from external device(s) / APIs 112, or other data sources that provide insight into the individual’s health-related beliefs.

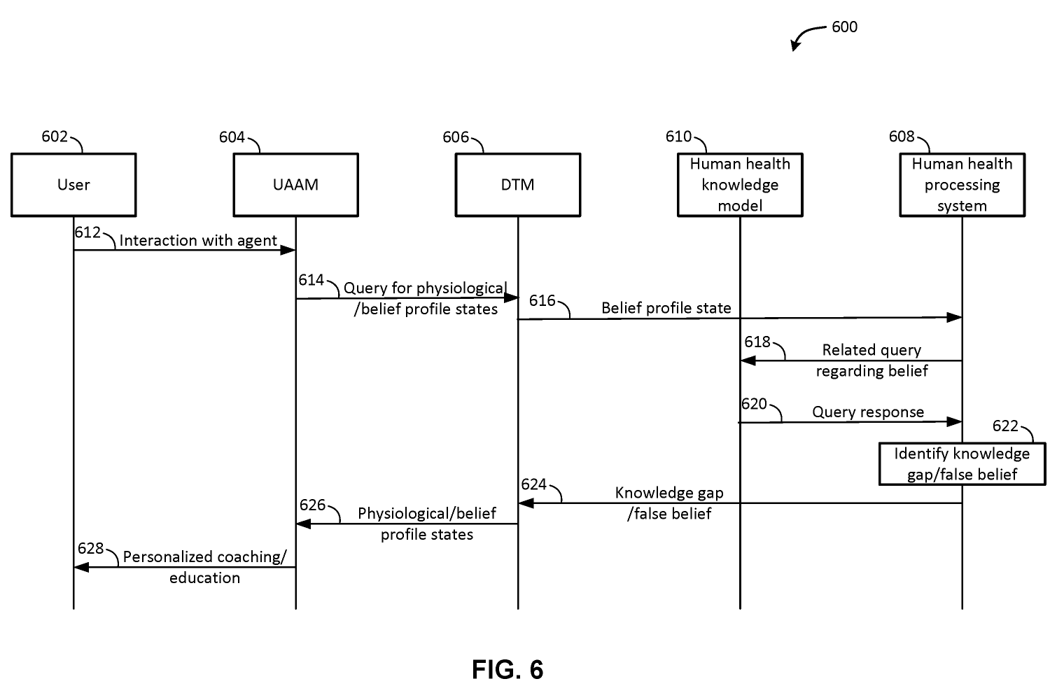

The patient belief profile 320 may represent the health-related beliefs of the individual in a structured format. The patient belief profile 320 may be stored as a structured database, a knowledge graph, a collection of belief statements with associated metadata, or as a machine learning model trained on the individual’s language and behavior patterns. The patient belief profile 320 may be analyzed by the human health processing system 104 shown in FIG. 1 to identify knowledge gaps or false beliefs by comparing the patient belief profile 320 against the human health knowledge model maintained by the human health knowledge model server 106. The comparison process and identification of knowledge gaps or false beliefs may be performed external to the patient belief profile structure 320 itself, as illustrated in FIG. 6 and discussed below.

The patient belief profile 320 may be updated continuously or periodically as new input data becomes available. As the individual interacts with the user-facing application 102, engages in new behaviors, or provides additional conversational input, the patient belief profile 320 may be refined to more accurately represent the individual’s current health-related beliefs. Historical beliefs may also be retained to better prepare the user-facing app agent module 204 to interact with the patient more meaningfully. The patient belief profile 320 may be used by the human health processing system 104 to personalize health interventions, educational content, and coaching delivered to the individual. For example, the user-facing app 102 may tell the patient, “you’ve come a long way since last summer when you used to think that a high protein diet low in fruits and vegetables was the way to go.”

In some embodiments, the patient belief profile 320 may mirror at least part of the organizational structure of the physiological model or the human health knowledge model, such that beliefs relating to nutrition, medication adherence, sleep, exercise, stress, symptoms, or other topics may be stored in corresponding branches or regions of the patient belief profile 320. Individual belief statements may be stored with timestamps, source identifiers, confidence values, and supporting evidence derived from user interactions 322, behavioral data 324, biometric data 326, or conversational data 328. Some portions of the patient belief profile 320 may represent uncertainty or absence of information where the system has insufficient evidence regarding the individual’s beliefs. The patient belief profile 320 may be updated over time by one or more background processes that reconcile newly observed statements or behaviors with previously stored belief data. In some embodiments, a discrepancy between the patient belief profile 320 and the human health knowledge model may be used not only for user-facing education, but additionally, or alternatively, to populate a clinician-facing flag or summary item for review through the clinician interface 804.

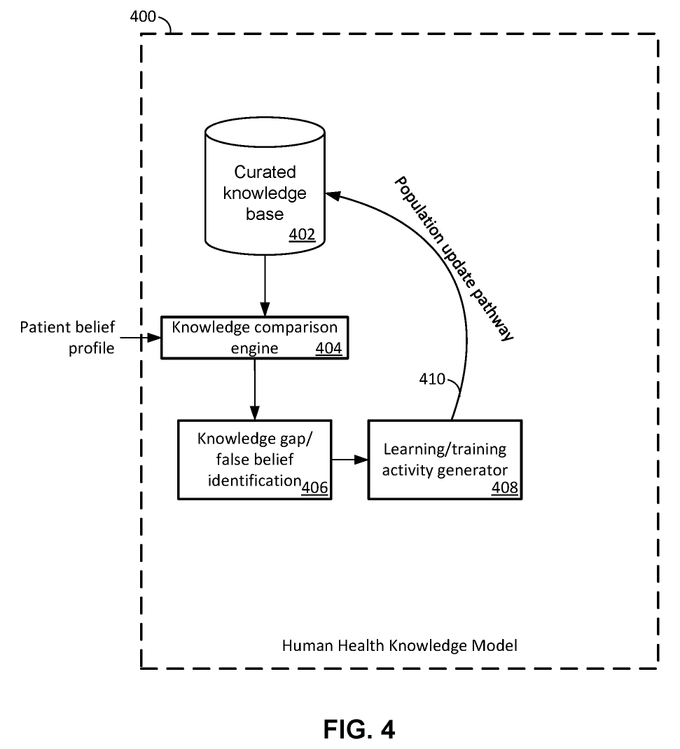

FIG. 4 illustrates a human health knowledge model structure 400 according to some embodiments. The human health knowledge model structure 400 may be provided as an example, and embodiments may vary in organization, content scope, and update mechanisms. The human health knowledge model structure 400 may represent the knowledge base maintained by the human health knowledge model server 106 shown in FIG. 1.

The human health knowledge model structure 400 may comprise a curated knowledge base 402 that contains scientifically validated information about human health. The curated knowledge base 402 may include information about human physiology, anatomical structures, biochemical processes, and physiological functions. The curated knowledge base 402 may further include information about interactions between the human body and external factors such as diet, exercise, pharmacological agents, environmental conditions, sleep, stress, and other lifestyle or environmental variables. The curated knowledge base 402 may be organized hierarchically, semantically, or using other structuring approaches to enable efficient retrieval and comparison operations. The curated knowledge base 402 may be implemented as a relational database, a graph database, a document store, or a knowledge graph with ontological relationships. In some examples the curated knowledge base 402 may be structured with similar structure to that of the digital twin 300 for humans or mirror components of the digital twin 300. For example, if blood pressure is increased in the circulatory/respiratory systems 304 the curated knowledge base 402 may have information regarding comorbidities with this condition.

The human health knowledge model structure 400 may further comprise a knowledge comparison engine 404 that compares patient belief profiles against the curated knowledge base 402. The knowledge comparison engine 404 may receive a patient belief profile, such as the patient belief profile 320 shown in FIG. 3B, as an external input. The knowledge comparison engine 404 may access the curated knowledge base 402 to retrieve relevant health information for comparison. The knowledge comparison engine 404 may analyze the beliefs represented in the patient belief profile and identify discrepancies, inconsistencies, or gaps relative to the curated knowledge base 402. The knowledge comparison engine 404 may be implemented using rule-based logic, semantic similarity algorithms, natural language processing models, or machine learning classifiers.

In some embodiments, the curated knowledge base 402 may represent health information using conditional, context-dependent, statistical, or non-binary relationships rather than fixed true-or-false statements. For example, a dietary recommendation may depend on a combination of metabolic state, medication status, activity level, time of day, pregnancy status, diagnosed conditions, or recent physiological trends. The knowledge comparison engine 404 may therefore determine that a belief is fully consistent, partially consistent, inconsistent under a current context, or unsupported by available information. The knowledge comparison engine 404 may additionally, or alternatively, generate a confidence value or uncertainty measure associated with a comparison result. In some embodiments, generative language processing may be used to formulate conversational explanations, while the underlying substantive content is constrained by the curated knowledge base 402.

The knowledge comparison engine 404 may generate a knowledge gap and false belief identification output 406. The output 406 may identify topics, concepts, or health-related information that are absent or incomplete in the patient belief profile, as well as beliefs held by the individual that contradict or are inconsistent with the curated knowledge base 402. The output 406 may be provided to a learning and training activity generator 408.

The learning and training activity generator 408 may receive the knowledge gap and false belief identification output 406 and generate targeted educational content, learning modules, training activities, or personalized explanations to address the identified gaps or false beliefs. The learning and training activity generator 408 may select content from a library of educational materials, generate custom explanations using generative AI, or create interactive tutorials tailored to the individual’s learning preferences and current knowledge state. The generated educational content may be delivered to the individual via the user-facing application 102 shown in FIG. 1.

In some embodiments, the learning and training activity generator 408 may tailor educational content to include information about potential adverse outcomes that may result from maintaining false beliefs or engaging in unhealthy behaviors. The educational content may present scientifically validated information about health risks in a manner that motivates behavior change without being demeaning or excessively alarming. For example, if an individual holds a false belief that a particular dietary pattern is healthy when evidence suggests it may increase disease risk, the educational content may explain the potential long-term consequences of maintaining that dietary pattern, while simultaneously providing clear guidance on healthier alternatives. The system may calibrate the tone and emphasis of risk-related messaging based on the individual's psychological profile, health literacy level, and responsiveness to different motivational approaches. Some individuals may respond more effectively to educational content that emphasizes potential adverse outcomes, while others may respond better to content that emphasizes positive benefits. The system may adapt its messaging strategy over time based on observed engagement and behavior change patterns for each individual.

In cases where the curated knowledge base 402 lacks information that is present in the patient belief profile 320, the system administrators who curate the knowledge base may be notified and these administrators after doing research into the topic may elect to update the curated knowledge base 402. For example, a clinical psychologist who has experienced PTSD and has bipolar 1 who is also a patient may have detailed beliefs about the relationship between parameters in the digital twin 300 and the curated knowledge base may lack information in this regard. For example, the patient may have beliefs about the relationships between mania and diet and sleep for those with bipolar 1 and PTSD, such as an increased risk of nightmares based on the type of food that was last ingested before bed. In another example, a personal trainer who is also a patient may have beliefs that certain exercises done in combination and following a dose of creatine when repeated over time in an individual with a particular health condition such as type 2 diabetes may have a particular effect on a seemingly unrelated part of the human body. In these cases, the system may respond to the patient that it is unsure about the details of those relationships. Then, the system 100 may flag for its administrators an area where the curated knowledge base 402 is incomplete. In this way, the knowledge comparison engine 404 may identify known gaps in knowledge of the patient based on credible aspects of the curated knowledge base 402 or it may identify gaps in knowledge of the curated knowledge base 402.

The human health knowledge model structure 400 may further comprise a population update pathway 410 that enables refinement of the curated knowledge base 402 based on aggregated data from multiple individuals. The population update pathway 410 may receive de-identified or anonymized data from digital twins stored in the digital twin database 108 shown in FIG. 1. The population update pathway 410 may analyze population-level trends, correlations, and outcomes to identify new insights, validate existing knowledge, or refine recommendations. For example, if aggregated data reveals that a particular dietary pattern consistently correlates with improved glycemic control across a population cohort, this insight may be incorporated into the curated knowledge base 402 via the population update pathway 410. The population update pathway 410 may enable continuous improvement of the human health knowledge model structure 400 as more individuals use the system and contribute data. The curated knowledge base 402 may be updated periodically by domain experts, automatically based on new scientific literature, or through the population update pathway 410 based on real-world data from the system’s user population.

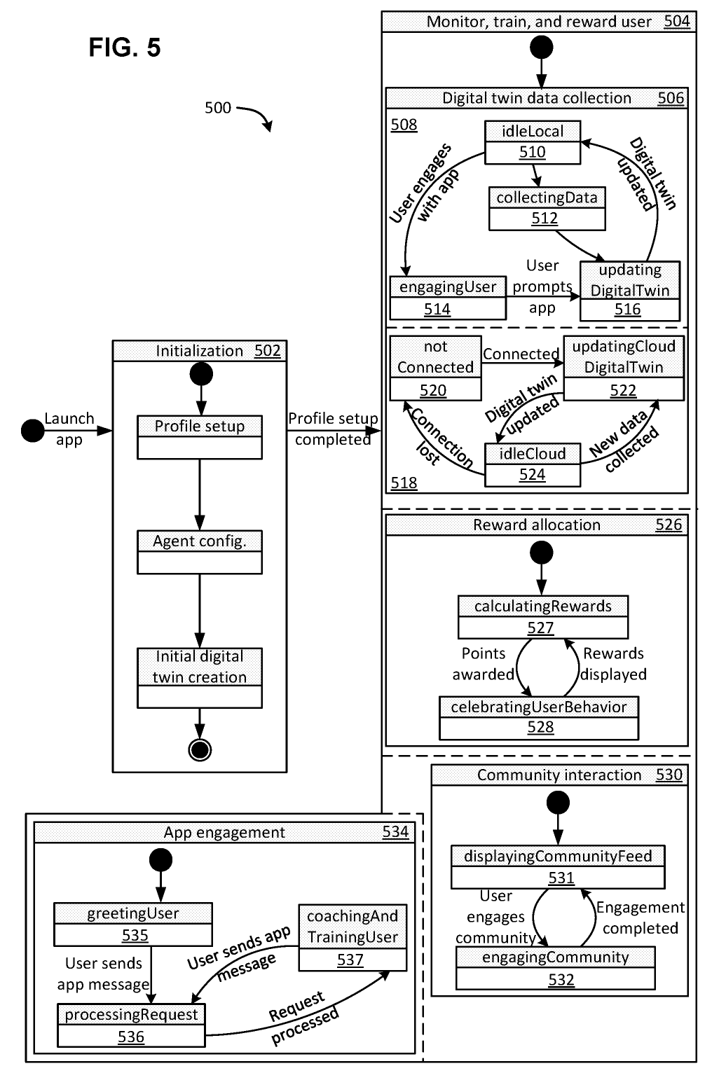

FIG. 5 illustrates a gamification state machine 500 according to some embodiments. The gamification state machine 500 may be provided as an example, and embodiments may vary in state definitions, transitions, and reward mechanisms. The gamification state machine 500 may represent the operational states and transitions of the gamification engine 212 shown in FIG. 2.

The gamification state machine 500 may begin with an initialization state 502. In the initialization state 502, the user-facing application may perform setup operations such as user profile creation, configuration of the user-facing app agent module, establishment of initial goals or preferences, and creation of an initial digital twin for the individual. Upon completion of initialization, the state machine 500 may transition to a composite state 504 for monitoring, training, and rewarding the user.

The composite state 504 may include multiple elements of the system running concurrently such as digital twin data collection 506, reward allocation 526, community interaction 530, and app engagement 534. In other examples, only one sub-state may be running at a time, and the system would transition from one substate to another. The composite state 504 may comprise a building twin data collection substate 506 that manages local data acquisition and cloud synchronization. The building twin data collection substate 506 may include two concurrent subsections that may operate in parallel.

A first subsection 508 may manage local data operations and may include an idle local state 510, a collecting data state 512, an engaging user state 514, and an updating digital twin state 516. The idle local state 510 may represent a standby condition in which the application is waiting for data input, user interaction, or scheduled events. The collecting data state 512 may represent active acquisition of behavioral data, biometric data, or other information from sensors, external devices, or user input. The engaging user state 514 may represent active interaction between the individual and the user-facing app agent module, such as conversational exchanges, coaching sessions, or delivery of educational content. The updating digital twin state 516 may represent updating of the local digital twin maintained by the digital twin module 208 based on newly collected data.

A second subsection 518 may manage cloud synchronization operations and may include a not connected state 520, an updating cloud digital twin state 522, and an idle cloud state 524. The not connected state 520 may represent a condition in which network connectivity to the remote digital twin database 108 is unavailable. The updating cloud digital twin state 522 may represent synchronization of the local digital twin with the remote digital twin database 108 via the cloud digital twin interface module 210 when network connectivity becomes available. The idle cloud state 524 may represent a condition in which the cloud synchronization is complete and the system is waiting for the next synchronization event or for new data to accumulate locally.

The gamification state machine 500 may further comprise a reward allocation state 526 that calculates and awards rewards to the individual based on detected healthy behaviors. The reward allocation state 526 may be triggered when the application detects that the individual has performed a healthy behavior, such as exercising, eating a nutritious meal, achieving a sleep goal, or completing a learning activity. The reward allocation state 526 may comprise two substates: a calculating rewards state 527 and a celebrating user behavior state 528. In the calculating rewards state 527, the application may calculate reward points or other incentives based on predefined criteria, dynamic goals, or personalized targets derived from the digital twin. Upon completion of the calculation, the state machine may transition from the calculating rewards state 527 to the celebrating user behavior state 528, with the transition labeled “Points awarded” to indicate that the reward calculation is complete and points have been assigned to the individual. In the celebrating user behavior state 528, the application may present the awarded rewards to the individual via the user interface module 202, displaying congratulatory messages, visual animations, or other celebratory feedback to reinforce the healthy behavior. Upon completion of the reward display, the state machine may transition from the celebrating user behavior state 528 back to the calculating rewards state 527, with the transition labeled “Rewards displayed” to indicate that the celebration is complete and the system is ready to calculate rewards for the next detected healthy behavior. In some examples, the rewards may include items of monetary value, or the points awarded may be exchanged for real-world goods or services or gift cards.

In some embodiments, the gamification state machine 500 may incorporate motivational elements that combine positive reinforcement with awareness of potential adverse outcomes. While the reward allocation state 526 provides positive feedback for healthy behaviors, the system may also present information about undesirable health outcomes that the individual has successfully avoided through their healthy behaviors. For example, when celebrating a user’s achievement of consistent exercise goals, the system may acknowledge both the positive benefits gained and the health risks that have been reduced or avoided. This dual approach may provide a more complete motivational framework that recognizes both the pursuit of positive outcomes and the avoidance of negative outcomes as valid and powerful motivators. The system may personalize the balance between these motivational approaches based on the individual’s psychological profile, with some individuals receiving more emphasis on avoided risks and others receiving more emphasis on achieved benefits. The celebration of risk avoidance may be framed in an empowering, positive manner that reinforces the individual’s agency and success in protecting their health.

The gamification state machine 500 may further comprise a community interaction state 530 that enables the individual to engage with community features such as a social feed, event postings, or shared content. The community interaction state 530 may comprise two substates: a displaying community feed state 531 and an engaging community state 532. In the displaying community feed state 531, the application may present community content to the individual via the user interface module 202, such as posts from other users, shared achievements, healthy recipes, community events, or other social content. When the individual chooses to interact with the community content, the state machine may transition from the displaying community feed state 531 to the engaging community state 532, with the transition labeled “User engages community” to indicate that the individual has initiated an interaction such as posting content, commenting, liking a post, or registering for an event. In the engaging community state 532, the application may process the individual’s community interaction, update the community feed, award any applicable rewards for community participation, and provide feedback to the individual. Upon completion of the engagement action, the state machine may transition from the engaging community state 532 back to the displaying community feed state 531, with the transition labeled “Engagement completed” to indicate that the interaction is complete and the community feed is ready to be displayed again. The community interaction state 530 may integrate with the community engagement module 214 shown in FIG. 2.

The composite state 504 may further comprise an app engagement state 534 that represents active interaction with the user-facing app agent module. The app engagement state 534 may begin with a greeting user state 535 in which the user-facing app agent module presents an initial greeting or welcome message to the individual upon opening the application or initiating a conversation. When the individual sends a message to the application, the state machine may transition from the greeting user state 535 to a processing request state 536, with the transition labeled “User sends app message” to indicate that the individual has provided input that requires processing. Here, a “message” is defined as any information being sent to or from the user and to or from the system and may include text, voice to text, voice messages, video messages, or similar. In the processing request state 536, the application may analyze the individual’s message, determine the intent or request, retrieve relevant information from the digital twin or patient belief profile, and prepare an appropriate response. Upon completion of request processing, the state machine may transition from the processing request state 536 to a coaching and training user state 537, with the transition labeled “Request processed” to indicate that the application is ready to deliver a response. In the coaching and training user state 537, the user-facing app agent module may deliver personalized coaching, educational content, health recommendations, or other responses based on the processed request and the individual’s digital twin and patient belief profile. If the individual sends another message during the coaching interaction, the state machine may transition from the coaching and training user state 537 back to the processing request state 536, with the transition labeled “User sends app message” to indicate that a new user input has been received and requires processing. This cycle may continue as the individual engages in an ongoing conversation or interaction with the user-facing app agent module. The app engagement state 534 may integrate with the user-facing app agent module 204 shown in FIG. 2.

FIG. 6 illustrates a message flow diagram 600 for identifying knowledge gaps or false beliefs and delivering personalized coaching and education according to some embodiments. The message flow diagram 600 may be provided as an example, and embodiments may vary in the specific messages exchanged, the sequence of interactions, or the system components involved. The message flow diagram 600 may represent an example interaction flow when an individual engages with the user-facing app agent module and the system identifies an opportunity to deliver educational content based on a knowledge gap or false belief.

The message flow diagram 600 may involve several entities. A user 602 may represent the individual interacting with the system. A user-facing app agent module (UAAM) 604 may represent the conversational agent or coaching interface, corresponding to the user-facing app agent module 204 shown in FIG. 2. A digital twin module (DTM) 606 may represent the local digital twin storage and management component, corresponding to the digital twin module 208 shown in FIG. 2. A human health processing system 608 may represent the backend processing system, corresponding to the human health processing system 104 shown in FIG. 1. A human health knowledge model 610 may represent the curated knowledge base, corresponding to the human health knowledge model server 106 shown in FIG. 1.

The message flow may begin when the user 602 initiates an interaction with the UAAM 604 as shown by arrow 612. The interaction may comprise a conversational message, a question, a request for coaching, or other user input, for example. In response to the user interaction, the UAAM 604 may query the DTM 606 for current physiological and belief profile states, as shown by arrow 614. The DTM 606 may retrieve the relevant data from the digital twin and patient belief profile. The DTM 606 may transmit the belief profile state to the human health processing system 608, as shown by arrow 616. As noted, the belief profile state may include information about the individual’s current health-related beliefs, knowledge level, or expressed understanding of health topics relevant to the user’s interaction. In some examples, the belief profile may be separate from the DTM 606. The human health processing system 608 may formulate a query to the human health knowledge model 610 to retrieve scientifically validated information related to the belief expressed by the user. This query may be indicated by an arrow 618. The human health knowledge model 610 may process the query and retrieve relevant information from the curated knowledge base. The human health knowledge model 610 may return a query response to the human health processing system 608, indicated by arrow 620. The query response may include validated health information, evidence-based recommendations, or other content from the curated knowledge base.

In an alternative embodiment, the data flow for updating and utilizing the patient belief profile may be different. The user-facing app agent module (UAAM) 604 may interface directly with the human health processing system 608 to provide updates on the individual’s beliefs or to request analysis. In this arrangement, the human health processing system 608 may then interface with the digital twin module (DTM) 606 and/or the cloud digital twin interface module 610 to retrieve the current state of the digital twin or other relevant physiological data. After processing this information, the human health processing system 608 may provide responsive information, such as identified knowledge gaps or new educational content, back to the UAAM 604 for delivery to the individual.

The human health processing system 608 may perform a comparison between the belief profile state received from the DTM 606 and the query response received from the human health knowledge model 610. This comparison process may be indicated by block 622. The human health processing system 608 may determine whether the individual’s belief is consistent with the validated knowledge, or whether a knowledge gap or false belief exists. If a knowledge gap or false belief is identified, the human health processing system 608 may transmit this information to the DTM 606, indicated by arrow 624. The DTM 606 may update the patient belief profile to reflect the identified gap or false belief. The DTM 606 may store the updated belief profile state as parametric data within the digital twin, maintaining models, data, and simulation parameters. The DTM 606 may return the physiological and belief profile states, including the identified knowledge gap or false belief, to the UAAM 604, indicated by arrow 626. The UAAM 604 may use this information to generate a personalized response. The UAAM 604 may deliver personalized coaching and education to the user 602, indicated by arrow 628. The personalized coaching and education may address the identified knowledge gap or false belief, provide scientifically validated information, offer behavior change recommendations, or deliver other educational content tailored to the individual’s current understanding and health state.

In some embodiments, the personalized coaching and education delivered by the UAAM 604 may incorporate motivational messaging that leverages awareness of potential adverse health outcomes. When appropriate for the individual’s psychological profile and current health state, the coaching may include information about health risks or undesirable physiological consequences that may result from current behaviors or beliefs. The messaging may be framed in a supportive, non-judgmental manner that emphasizes the individual’s ability to avoid these outcomes through specific behavior changes. For example, the UAAM 604 may inform an individual that their current activity level and dietary patterns, if continued, may increase their risk of developing certain health conditions, while simultaneously providing a personalized action plan to reduce that risk. The system may balance risk-awareness messaging with positive reinforcement and encouragement, recognizing that for some individuals, understanding what they are working to avoid may be as motivating as understanding what they are working to achieve. The UAAM 604 may monitor the individual’s emotional and behavioral responses to different messaging approaches and may adjust the balance between risk-focused and benefit-focused messaging to optimize engagement and behavior change outcomes.

Through this message flow, the system may identify discrepancies between an individual’s health-related beliefs and validated scientific knowledge, and may deliver targeted educational interventions to improve health literacy and support informed decision-making. The message flow diagram 600 may represent one interaction cycle, and may be repeated as the user continues to engage with the user-facing app agent module over time. Other architectures are possible as well such as the UAAM 604 interfacing with the Human health processing system 608 which then interfaces with the DTM 606 and Human health knowledge model 610 as required. As noted, in some embodiments the patient belief profile may be implemented as part of the DTM 606 or as a separate module.

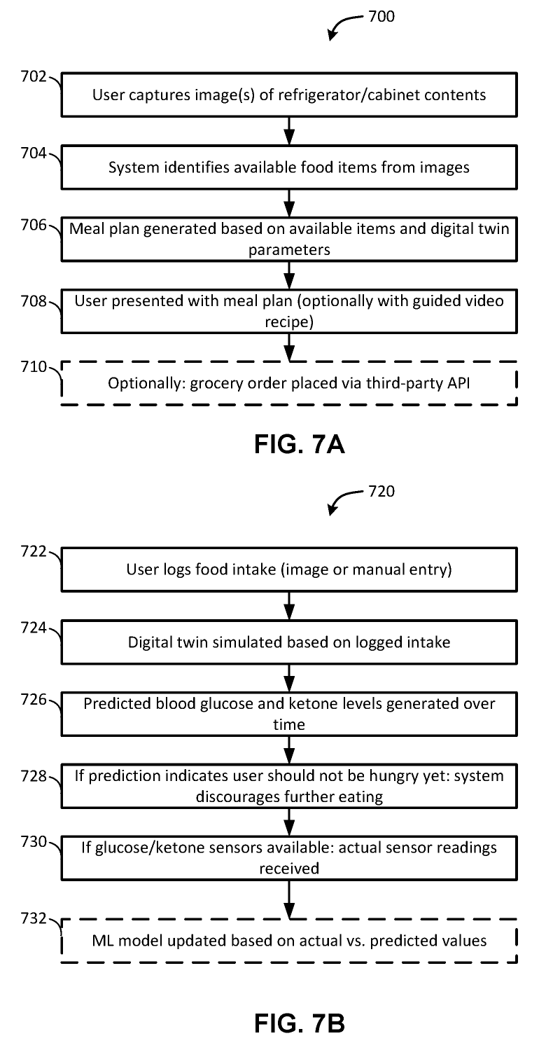

FIG. 7A illustrates a meal planning workflow 700 according to some embodiments. The meal planning workflow 700 may be provided as an example, and embodiments may vary in the specific steps, user interactions, or system processing involved. The meal planning workflow 700 may represent functionality provided by the user-facing application 102, which may utilize the user-facing app agent module 204, the digital twin module 208, the human health processing system 104, and third-party APIs 218. Ultimately, the meal planning workflow 700 may enable convenient, personalized meal planning that takes into account the user’s available food resources, current physiological state, and health goals, thereby supporting healthier eating behaviors and improved health outcomes.

The meal planning workflow 700 may begin at block 702, where the user captures images of refrigerator or cabinet contents. The user may use a camera on a mobile device executing the user-facing application 102 to capture images of available food items stored in a refrigerator, pantry, cabinets, or other food storage locations. The captured images may be transmitted to the user-facing application 102 for processing.

At block 704, the system identifies available food items from the images. Image processing may be performed locally on the user device by the user-facing app agent module, remotely by the human health processing system 104, or using a combination of local and cloud-based processing. The system may utilize image recognition algorithms, machine learning models trained on food images, or computer vision techniques to detect and classify food items visible in the images. The system may generate a list or inventory of identified food items, which may include item names, quantities, estimated freshness, or nutritional information.

At block 706, a meal plan is generated based on available items and digital twin parameters. The system may access the digital twin to retrieve physiological parameters, dietary preferences, health goals, restrictions, or other relevant information about the user. The digital twin may be stored locally on the user device via the digital twin module 208, remotely in the digital twin database 108, or synchronized between local and cloud storage. Meal plan generation may be performed by the user-facing app agent module executing locally on the user device, by the human health processing system 104 in the cloud, or by a combination of local and remote processing. The meal plan may include one or more meal suggestions, recipes, portion sizes, preparation instructions, or nutritional information. The meal plan may be optimized to support the user’s health goals, such as maintaining stable blood glucose levels, achieving a target caloric intake, or meeting macronutrient targets.

At block 708, the user is presented with the meal plan, optionally with a guided video recipe. The system may present the generated meal plan to the user via the user interface module 202. The meal plan may be displayed as text, images, videos, or interactive content. If a guided video recipe is available, the system may present step-by-step video instructions for preparing a selected meal from the meal plan. The user may review the meal plan, select a preferred meal option, request modifications, or accept the plan as presented.

At block 710, optionally a grocery order is placed via a third party API 218. If certain ingredients are not present in the captured images but are required for a recommended recipe, the system may identify missing ingredients and may invoke third-party APIs 218 to facilitate grocery ordering. The third-party APIs may include grocery delivery services, online retailers, or meal kit providers. The system may automatically place an order for missing ingredients on behalf of the user, or may present the user with an option to approve and complete the order.

FIG. 7B illustrates a food logging and digital twin simulation workflow 720 according to some embodiments. The food logging and digital twin simulation workflow 720 may be provided as an example, and embodiments may vary in the specific steps, data sources, or simulation techniques involved. The food logging and digital twin simulation workflow 720 may represent functionality provided by the user-facing application 102, which may utilize the user-facing app agent module 204, the digital twin module 208, and the human health processing system 104. The food logging and digital twin simulation workflow 720 may enable users to understand the physiological impact of their food choices in real time, supporting more informed decision-making and healthier eating behaviors. By continuously refining the digital twin based on actual sensor data, the system may provide increasingly accurate and personalized predictions over time.

The food logging and digital twin simulation workflow 720 may begin at block 722, where the user logs food intake, either via image or manual entry. The user may capture an image of a meal or food item using a camera on a mobile device executing the user-facing application 102, or may manually enter food information via the user interface module 202. The logged food intake information may be transmitted to the system for processing.

At block 724, a digital twin is simulated based on logged intake. The system may process the logged food intake to identify the food items consumed, estimate portion sizes, and determine nutritional content such as calories, carbohydrates, proteins, fats, fiber, and micronutrients. The system may utilize image recognition, nutritional databases, or machine learning models to analyze the food intake data. The digital twin module 208 or the human health processing system 104 may simulate the digital twin based on the logged food intake to generate predicted physiological outcomes.

At block 726, predicted blood glucose and ketone levels are generated over time. The simulation may predict how the individual’s blood glucose levels, ketone levels, insulin response, energy levels, timing of future bowel movements, or other physiological parameters are expected to change over time in response to the consumed food. The predicted outcome may be generated as a time-series curve, numerical values, or other representation showing the expected physiological response over the next several hours.

At block 728, if the prediction indicates the user should not be hungry yet, the system discourages further eating. The system may analyze the predicted physiological outcome to determine whether the user’s blood glucose levels are expected to remain elevated, whether the user has consumed sufficient calories, or whether other indicators suggest that further eating is not advisable at the current time. If the prediction indicates that the user should not be hungry yet or that further eating may lead to excessive glucose elevation or other adverse outcomes, the system may present a message to the user via the user interface module 202 discouraging additional food intake and may suggest waiting or choosing a lower-glycemic snack or beverage such as tea. In some cases, the system may encourage unlimited consumption of certain foods such as low caloric density vegetables in case someone has a high level of hunger when discouraged from eating more.

At block 730, if glucose or ketone sensors are available, actual sensor readings are received. If the user has access to biometric sensors such as a continuous glucose monitor (CGM), ketone meter, or other physiological monitoring devices, the system may receive actual sensor readings after the meal. The actual sensor readings may be transmitted to the system via the data acquisition module 206 or external devices and APIs 112.

At block 732, a machine learning model is updated based on actual versus predicted values. The system may compare the actual sensor readings to the predicted physiological outcome generated at block 726 to assess the accuracy of the digital twin simulation. The digital twin module 208 or the human health processing system 104 may update or refine the digital twin model, machine learning models, or simulation parameters based on the comparison between predicted and actual values. This feedback loop may enable continuous personalization and calibration of the digital twin to the individual’s unique physiological responses, improving the accuracy of future predictions.

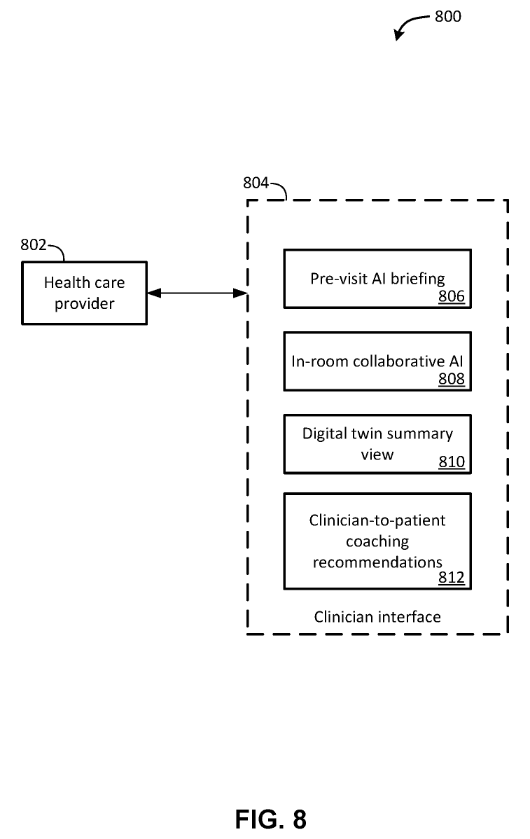

FIG. 8 illustrates a clinician interface system 800 according to some embodiments. The clinician interface system 800 may be provided as an example, and embodiments may vary in the specific features, modules, or user interface elements provided. The clinician interface system 800 may represent the clinician interface 114 shown in FIG. 1, and may enable healthcare providers to review patient data, access digital twin information, and deliver care.

The clinician interface system 800 may facilitate interaction between a healthcare provider 802 and a clinician interface 804. The healthcare provider 802 may represent a physician, nurse, physician assistant, care coordinator, or other clinical user who interacts with the system to review patient information and provide clinical guidance. The clinician interface 804 may be implemented as a web-based portal, a desktop application, or a mobile application accessible by the healthcare provider 802.

The clinician interface 804 may comprise several integrated modules or features. A pre-visit AI briefing module 806 may generate and present a summary of patient information prior to a clinical encounter. The pre-visit AI briefing may retrieve data from the digital twin database 108 and the patient belief profiles 105 shown in FIG. 1, and may use artificial intelligence or machine learning models to synthesize the information into a concise summary. The pre-visit AI briefing may include information about the patient’s current physiological state, recent behavioral patterns, identified knowledge gaps or false beliefs, medication adherence, recent alerts or anomalies detected by the home-based monitoring subsystem, and other relevant clinical information. The healthcare provider 802 may review the pre-visit AI briefing before meeting with the patient to prepare for the encounter and identify topics to address during the visit.

An in-room collaborative AI module 808 may provide real-time assistance during the clinical encounter. The in-room collaborative AI may transcribe the conversation between the healthcare provider 802 and the patient, generate structured clinical notes, suggest diagnostic codes or billing codes, prepare medication orders or laboratory orders for review and approval by the healthcare provider, and provide clinical decision support based on the patient’s digital twin and current symptoms or complaints. The in-room collaborative AI may reduce documentation burden on the healthcare provider 802 and enable more time for direct patient interaction.

A digital twin summary view module 810 may present a visual and textual summary of the patient’s digital twin. The digital twin summary view may display physiological parameters from the digital twin structure 300 shown in FIG. 3A, the patient belief profile 320 shown in FIG. 3B, trends over time in certain health metrics, identified knowledge gaps or false beliefs, and comparisons to population baselines or clinical targets. The healthcare provider 802 may interact with the digital twin summary view to explore specific aspects of the patient’s health state, review historical data, or assess the impact of prior interventions.

In some embodiments, the digital twin summary view module 810 may present information at multiple levels of detail. A first view may present a compact clinical summary using status indicators, such as color-coded indicators, icons, scores, or ranked issue lists, to identify topics for attention. A second view may provide drill-down access to underlying data, including recent values, averages, ranges, trends over time, activity timelines, inferred physiological parameters, belief-profile evidence, population comparisons, and confidence or uncertainty information associated with a displayed value or prediction. The clinician interface 804 may additionally, or alternatively, present forward-looking outputs generated from simulation of the digital twin, including predicted near-term health states, predicted response to a behavior change, or predicted response to a treatment adjustment. In some embodiments, the clinician interface 804 may expose more detailed data and analytical views than a patient-facing interface, while the patient-facing interface may present a simplified representation of selected outputs.

A clinician-to-patient coaching recommendations module 812 may enable the healthcare provider 802 to input recommendations, guidance, or educational content to be delivered to the patient via the user-facing application 102. The healthcare provider 802 may specify behaviors to encourage, topics for the user-facing app agent module to address in future interactions, educational content to deliver, or goals for the patient to work toward. The clinician input may include lifestyle prescriptions that specify target behaviors for the individual, such as a target amount of daily physical activity, a target dietary composition, a sleep schedule, a hydration target, a stress management routine, or other behavioral goals. The clinician input may additionally or alternatively include medication prescriptions, such as prescribed medications, dosages, administration schedules, refill instructions, or other pharmacological directives. The system may incorporate medication prescriptions into the personalized health interventions by providing medication reminders, tracking medication adherence, delivering educational content about prescribed medications, or alerting the individual to potential interactions between medications and observed behaviors or dietary patterns reflected in the digital twin. In some examples, the clinician may enter a range of acceptable dosage of a medication, such as 50 mg to 150 mg of trazodone for sleep. However, in this example the system may deliver personalized recommendations on a nightly basis for how much to take based on the information stored in the digital twin for the day. For example, if someone is actively losing weight and had a significant caloric deficit, the system may recommend the full 150 mg of trazodone whereas if someone had a large caloric intake that day and heart rate variability indicated it was a low stress day, it may only recommend 50 mg. The clinician-to-patient coaching recommendations may be transmitted to the human health processing system 104 and incorporated into the personalized health interventions delivered to the patient via the user-facing application 102. The clinician input may be surfaced to the individual through multiple channels of the user-facing application 102, including a dedicated page or area of the user interface module 202, conversational messaging delivered by the user-facing app agent module 204, reward criteria within the gamification engine 212, or combinations thereof. In some embodiments, the individual may use the system without clinician involvement, and the clinician-to-patient coaching recommendations module 812 may remain inactive or unused until a healthcare provider elects to provide input for the individual.

The clinician interface 804 may communicate with the human health processing system 104 and the digital twin database 108 via the network 103 shown in FIG. 1. The clinician interface 804 may retrieve patient data, update clinical records, and transmit clinician input back to the system for incorporation into the patient’s digital twin and patient belief profile. Through the clinician interface 804, healthcare providers may leverage the comprehensive data collected by the system to provide more informed, personalized, and effective care.

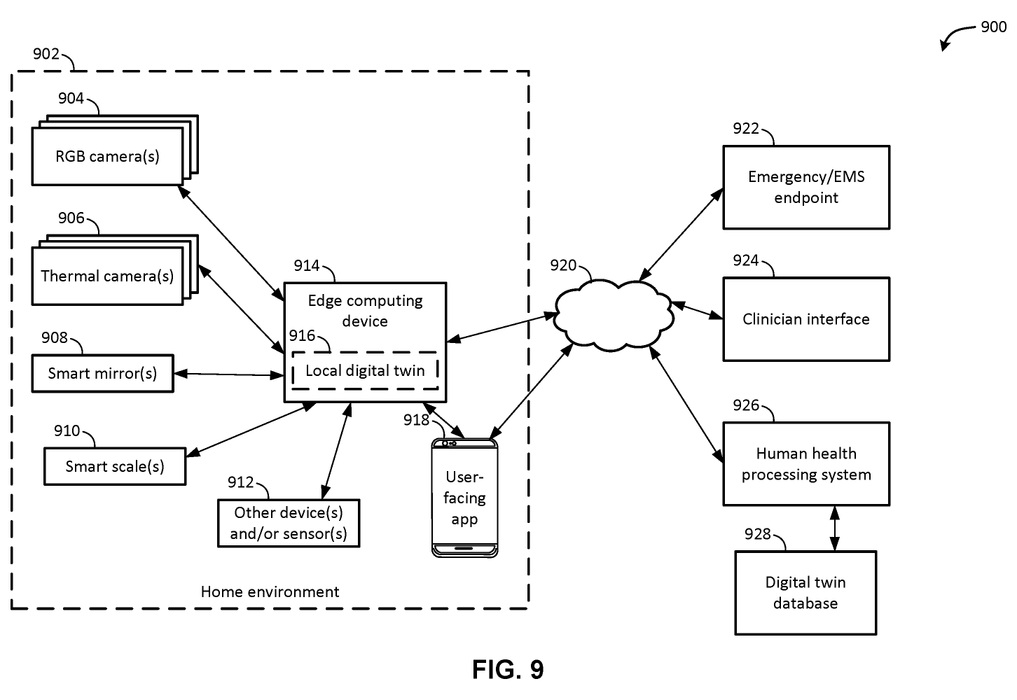

FIG. 9 illustrates a home-based remote monitoring system architecture 900 according to some embodiments. The home-based remote monitoring system architecture 900 may be provided as an example, and embodiments may vary in the specific sensors, devices, processing configurations, or external system connections involved. Arrows represent communication links, which may be direct or indirect (e.g., communication may be relayed via one or more other devices). The home-based remote monitoring system architecture 900 may represent the home-based remote monitoring subsystem 110 shown in FIG. 1, and may enable continuous monitoring of an individual within a home environment while preserving privacy through local processing.

The home-based remote monitoring system architecture 900 may comprise a home environment 902. The home environment 902 may represent a residence, apartment, assisted living facility, or other living space where an individual resides. Inside the home environment 902, several sensor and device components may be deployed to monitor the individual’s activities and physiological state.

One or more RGB cameras 904 may be positioned to capture visual image data of the individual within the home environment. The RGB cameras may be mounted on walls, ceilings, or furniture, or may be integrated into other devices such as smart displays or security systems. The RGB cameras may capture video frames continuously, periodically, or in response to detected motion or other triggers.

One or more thermal cameras 906 may be positioned to capture thermal image data of the individual. The thermal cameras may detect infrared radiation emitted by the individual’s body and may generate thermal images representing skin temperature distribution. The thermal cameras may be used to estimate physiological parameters such as body temperature, detect fever, or correlate thermal patterns with metabolic or glycemic states.

The thermal cameras 906 may additionally, or alternatively, support monitoring during periods in which pose changes or gross motion provide limited information, such as during sleep, rest, or prolonged inactivity. Thermal image data may be analyzed to detect temperature distributions over exposed skin, breathing-related thermal changes near the nose or mouth, bed occupancy patterns, or localized thermal anomalies. In some embodiments, thermal features may be used to infer respiratory activity, identify possible fever states, detect physiological stress, or improve estimation of glycemic events when movement-based observations are weak. Thermal processing may be fused with RGB-derived outputs or may be used independently when RGB data is unavailable or less informative.

One or more smart mirrors 908 may be deployed within the home environment. A smart mirror may comprise a display integrated into a mirror surface, enabling the presentation of information to the individual while they view their reflection. The smart mirror may display alerts, health status indicators, reminders, or other information generated by the system. The smart mirror may also include integrated cameras or sensors for capturing image data or biometric measurements.

The smart mirror 908 may additionally, or alternatively, provide a patient-facing interface for daily interaction with the system. The smart mirror 908 may present a daily summary including one or more of current health status indicators, medication reminders, sleep quality information, hydration guidance, meal guidance, scheduled activities, and alerts generated from the digital twin or the home-based remote monitoring subsystem. The smart mirror 908 may receive user input through touch input, voice input, gesture input, gaze tracking, or combinations thereof. In some embodiments, the smart mirror 908 may include one or more embedded RGB cameras, depth cameras, thermal cameras, microphones, speakers, or biometric sensors. The smart mirror 908 may additionally, or alternatively, function as an alternative to the user-facing application 918 for presentation of personalized coaching, educational content, and acknowledgements of alerts. For example, when the individual approaches the smart mirror 908 in the morning, the smart mirror 908 may present a summary of overnight sleep data, current physiological trends, pending medications, and a recommended breakfast selected based on the digital twin.

One or more smart scales 910 may be positioned within the home environment, typically in a bathroom or bedroom. A smart scale may measure body weight, body composition, body fat percentage, hydration level, visceral fat level, or other metrics when the individual steps onto the scale. The smart scale may transmit measurements to the edge computing device for incorporation into the digital twin.

Other devices and/or sensors 912 may be deployed within the home environment. These may include depth sensing cameras, microphones, speakers, environmental sensors (temperature, humidity, air quality), motion sensors, door/window sensors, smart appliances, wearable devices, continuous glucose monitors, blood pressure monitors, or other health-related sensors. The depth sensing cameras may capture three-dimensional spatial information to improve pose estimation, detect falls, measure gait characteristics, or assess body composition. The microphones may capture audio data for voice interaction, detection of respiratory sounds, identification of distress vocalizations, or monitoring of environmental audio cues. The speakers may provide audio output for alerts, reminders, coaching messages, guided meditation, or other auditory feedback to the individual. The other devices and/or sensors may provide additional data streams to enhance monitoring capabilities.

In some embodiments, a lighter-weight deployment may omit dedicated home-installed cameras or other fixed monitoring hardware. Additionally, or alternatively, image data, audio data, motion data, or other sensor data may be captured by a smartphone, tablet, laptop, wearable device, or smart display used by the individual. For example, the data acquisition module 206 may use a camera or microphone of a mobile device to obtain images of meals, body posture, facial appearance, breathing-related signals, voice characteristics, or other observables for incorporation into the digital twin. A dedicated home-based monitoring subsystem 110 may be deployed for individuals requiring additional oversight, while an application-centric embodiment may be used for other individuals.

An edge computing device 914 may be positioned within the home environment to serve as the central processing hub for the monitoring system. The edge computing device may comprise a dedicated hardware appliance, a general-purpose computer configured with monitoring software, a smart home hub, or other computing hardware capable of processing sensor data locally. The edge computing device may receive data feeds from the RGB cameras 904, thermal cameras 906, smart mirrors 908, smart scales 910, and other devices and/or sensors 912. The edge computing device may process the received data locally within the home environment to generate derived outputs such as activity timelines, inferred physiological parameters, and risk scores.

The edge computing device 914 optionally may maintain a local digital twin 916. The local digital twin may store a subset of the individual’s digital twin data locally on the edge computing device for rapid access and offline processing. The local digital twin may be synchronized with the remote digital twin database 928 when network connectivity is available. Additionally, or alternatively (e.g., depending on the processing capabilities of the edge computing device 914), the local digital twin 916 may comprise a partial or complete copy of the digital twin of an individual, capable of partial or complete modeling of the various physiological systems modeled by the digital twin.

A user-facing application 918 may be accessible to the individual via a mobile device, tablet, or computer. The user-facing application may correspond to the user-facing application 102 shown in FIG. 1. It may receive alerts, status updates, and other information from the edge computing device 914. The user-facing application 918 may also transmit user input, acknowledgments, or configuration settings back to the edge computing device 914.

A network or cloud connection 920 may enable communication between components within the home environment 902 and external systems. The network may comprise the Internet, a cellular network, a local area network, a wide area network, or any combination thereof. The edge computing device 914 and the user-facing application 918 may communicate with external systems via the network.

An emergency or EMS endpoint 922 may receive escalated alerts from the edge computing device 914 via the network 920. The emergency or EMS endpoint may correspond to the emergency services endpoint 116 shown in FIG. 1. If the edge computing device detects a high-risk anomaly and the individual is unresponsive, the system may transmit an alert to the emergency or EMS endpoint to request immediate medical assistance. If the edge computing device 914 detects that an individual is a risk to themselves or others, this may also alert emergency or EMS services 922 or alert trusted family members or friends according to clinician and patient settings or preferences.

A clinician interface 924 may enable healthcare providers to review monitoring data, timelines, and alerts generated by the home-based remote monitoring system. The clinician interface may correspond to the clinician interface 114 shown in FIG. 1. Healthcare providers, for example, may access the clinician interface via the network 920 to monitor patients remotely and respond to alerts.

A human health processing system 926 may perform data processing, analysis, and intervention generation functions. The human health processing system may correspond to the human health processing system 104 shown in FIG. 1. The human health processing system may receive derived outputs from the edge computing device 914 via the network 920 and may incorporate the derived outputs into the digital twin.

A digital twin database 928 may store digital twins for one or more individuals. The digital twin database may correspond to the digital twin database 108 shown in FIG. 1. The digital twin database may receive updates from the edge computing device 914 via the network 920, and may provide digital twin data to the human health processing system 926 for analysis and intervention generation.

One feature that may be included in the home-based remote monitoring system architecture 900, according to some embodiments, is a privacy boundary maintained at the edge of the home environment 902. Raw image data captured by the RGB cameras 904 and thermal cameras 906 may be processed locally by the edge computing device 914 and deleted after processing, such that no raw image data is transmitted outside the home environment 902 via the network 920. Only derived outputs such as activity labels, inferred physiological parameters, timelines, and risk scores may be transmitted to external systems, preserving the individual’s privacy while enabling remote monitoring and clinical oversight.

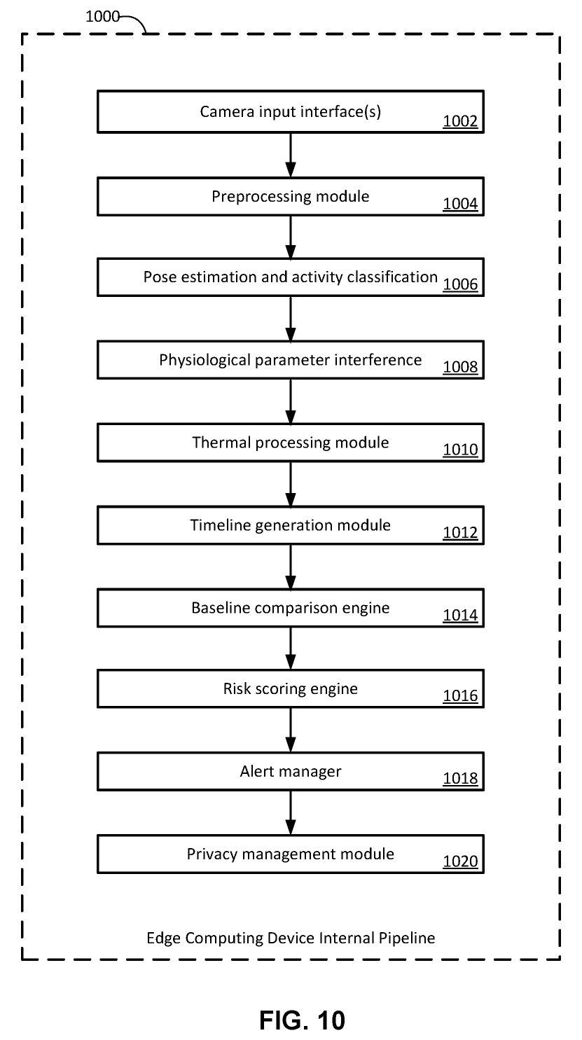

FIG. 10 illustrates an example processing architecture 1000 for an edge computing device according to some embodiments. The internal processing architecture 1000 may be provided as an example, and embodiments may vary in the specific modules, processing arrangements, or data flows involved. The internal processing architecture 1000 may represent the internal processing architecture of the edge computing device 914 shown in FIG. 9.

The internal processing architecture 1000 illustrated in FIG. 10 represents one example implementation, and the functionality described may be performed by different modules, components, or processing units in alternative embodiments. The specific allocation of functions to the modules shown in FIG. 10 is illustrative and not limiting. In some embodiments, multiple modules may be combined into a single processing unit, or a single module may be distributed across multiple processing units. Although illustrated as a sequence, the order of implementation of items within the internal processing architecture 1000 may vary. Data flow between the modules may be sequential for some functions, and may be parallel, asynchronous, branched, iterative, event-driven, or feedback-based for other functions. Some modules may operate continuously, periodically, on demand, or in response to detected conditions. The processing architecture may be implemented using dedicated hardware modules, software modules executing on general-purpose processors, field-programmable gate arrays (FPGAs), application-specific integrated circuits (ASICs), graphics processing units (GPUs), or any combination thereof.

The internal processing architecture 1000 may include camera input interfaces 1002 that receive video feeds from one or more RGB cameras 904 and/or thermal cameras 906 shown in FIG. 9. The camera input interfaces 1002 may support various video formats, frame rates, and resolutions, and may buffer incoming video frames for processing.

A preprocessing module 1004 may receive raw video frames from the camera input interfaces 1002 and perform initial processing operations. The preprocessing module may apply frame normalization, noise reduction, color correction, resolution scaling, or other image enhancement techniques to prepare the video frames for analysis by downstream modules.

A pose estimation and activity classification module 1006 may receive preprocessed video frames from the preprocessing module 1004 and apply a first machine learning model to detect the pose of the individual and classify the individual’s activity. The pose estimation may identify skeletal joint positions, body orientation, and posture. The activity classification may categorize the individual’s current activity into predefined classes such as sleeping, walking, standing, sitting, cooking, eating, exercising, or other activities.

A physiological parameter inference module 1008 may receive outputs from the pose estimation and activity classification module 1006 and apply a second machine learning model to infer one or more physiological parameters of the individual. The inferred physiological parameters may include heart rate, respiratory rate, body mass index, body fat percentage, glycemic risk, or other health-related metrics. The second machine learning model may be trained using synthetic training data generated by a physiological body model. In households with more than one individual, the physiological parameter inference module 1008 may utilize facial recognition, body recognition, or other biometric identification techniques to associate the inferred physiological parameters with the correct individual. The facial recognition may analyze facial features extracted from RGB camera data. The body recognition may analyze body shape, gait patterns, height, build, or other distinguishing physical characteristics to differentiate between household members. The identified individual’s physiological parameters may then be stored in association with that individual’s digital twin, ensuring that multi-person households can benefit from home-based monitoring without cross-contamination of health data between individuals.

In some embodiments, training data for the physiological parameter inference module 1008 may include synthetic data generated using a physiological body model. The physiological body model may simulate relationships between physiological states and externally observable image features, thermal signatures, motion patterns, body-shape changes, or temporal signal variations. Synthetic training data may be generated for different body types, clothing conditions, lighting conditions, camera placements, health states, ages, activity types, and environmental conditions. Additionally, or alternatively, the physiological parameter inference module 1008 may be trained or calibrated using clinical measurements, wearable sensor data, annotated human-observation data, or combinations thereof. Synthetic data may be used to supplement measured data, expand coverage of rare conditions, or improve robustness across sensing conditions.

The physiological parameter inference module 1008 may be implemented using different model architectures. In some embodiments, the physiological parameter inference module 1008 may receive features or intermediate outputs produced by the pose estimation and activity classification module 1006, as shown in FIG. 10. Additionally, or alternatively, a single machine learning model may receive image data and directly output activity classifications and inferred physiological parameters. Additionally, or alternatively, more than two machine learning models may be used, with different models specialized for different activities, camera modalities, physiological parameters, or patient populations. Model selection may be based on available compute resources, desired latency, target accuracy, power consumption, or the sensing configuration deployed in the home environment 902.

A thermal processing module 1010 may receive thermal image data from the camera input interfaces 1002 and process the thermal data to estimate skin temperature of the individual. The thermal processing module may correlate thermal patterns with physiological states such as fever, metabolic activity, or glycemic state. The thermal processing module may provide additional physiological parameter estimates that are fused with the outputs of the physiological parameter inference module 1008.

A timeline generation module 1012 may receive activity classifications and inferred physiological parameters from the pose estimation and activity classification module 1006, the physiological parameter inference module 1008, and the thermal processing module 1010. The timeline generation module may assemble a time-series representation of the individual’s activity states and physiological parameters over one or more time periods. The timeline generation module may implement adaptive time-step windowing, using shorter time windows during periods of active movement or detected anomalies and longer time windows during periods of inactivity or sleep.

A baseline comparison engine 1014 may receive the activity timeline from the timeline generation module 1012 and compare the current timeline against a personalized baseline of the individual. The personalized baseline may be established based on historical data collected over days, weeks, or months, and may represent the individual’s typical activity patterns and physiological parameter ranges. The baseline comparison engine may also compare the current timeline against population baselines derived from aggregated data from multiple individuals with similar characteristics. The baseline comparison engine may detect deviations or anomalies when the current timeline differs significantly from the personalized or population baselines.

A risk scoring engine 1016 may receive anomaly detection results from the baseline comparison engine 1014 and generate a risk score based on the detected anomalies. The risk score may quantify the likelihood or severity of an adverse health event, such as a hypoglycemic episode, a fall, or other medical emergency. The risk scoring engine may apply statistical models, rule-based logic, or machine learning classifiers to calculate the risk score. The risk score may be compared against one or more thresholds to determine whether an alert should be generated.

An alert transmission module 1018 may receive the risk score from the risk scoring module 1016 and may transmit an alert based on the risk score exceeding a threshold. The alert may be transmitted to emergency services, a designated caregiver, a clinician, family members, friends, neighbors, or other contacts. The alert transmission module 1018 may select the appropriate recipient or recipients based on the severity of the risk score, the type of detected event, the time of day, or other contextual factors. For example, a low-severity alert may be transmitted to a family member, while a high-severity alert may be transmitted to emergency services. In some embodiments, when a high-risk alert is generated, the alert transmission module 1018 may trigger automated actions to facilitate emergency response, such as unlocking doors to the house to enable emergency responders to gain entry without forced entry or delay. The door unlocking may be performed by transmitting a command to a smart lock system integrated with the home-based remote monitoring subsystem via an API. The alert may include information about the detected event, the individual’s current physiological state, the individual’s location within the home, and any other relevant context to assist the responder in providing appropriate assistance.

The alert manager 1018 may additionally, or alternatively, implement tiered routing logic based on severity, persistence, user responsiveness, and recipient role. A first alert may be transmitted to the individual through the user-facing application 918 or the smart mirror 908. If the individual does not acknowledge the alert within a threshold period, or if follow-up observations indicate increased risk, the alert manager 1018 may transmit a second alert to a caregiver, family member, neighbor, monitoring service, or healthcare provider. If the individual remains unresponsive or the risk score indicates an acute event, the alert manager 1018 may escalate the alert to the emergency or EMS endpoint 922. Different alert pathways may be used for different anomaly types, including fall events, glycemic events, respiratory anomalies, suicidal ideation, psychotic episodes, medication non-adherence, wandering behavior, prolonged inactivity, or other types.

A privacy management module 1020 may manage the deletion of raw image data after processing to preserve the individual’s privacy. The privacy management module may ensure that raw video frames received from the camera input interfaces 1002 are deleted from local storage after they have been processed by the preprocessing module 1004, the pose estimation and activity classification module 1006, the physiological parameter inference module 1008, and the thermal processing module 1010. The privacy management module may ensure that no raw image data is transmitted outside the home environment 902 via the network 920 shown in FIG. 9. Only derived outputs such as activity labels, inferred physiological parameters, timelines, and risk scores may be transmitted to external systems.

The internal processing architecture 1000 may operate continuously or periodically to monitor the individual within the home environment. The modular architecture may enable efficient processing, privacy preservation, and timely detection of health-related anomalies.

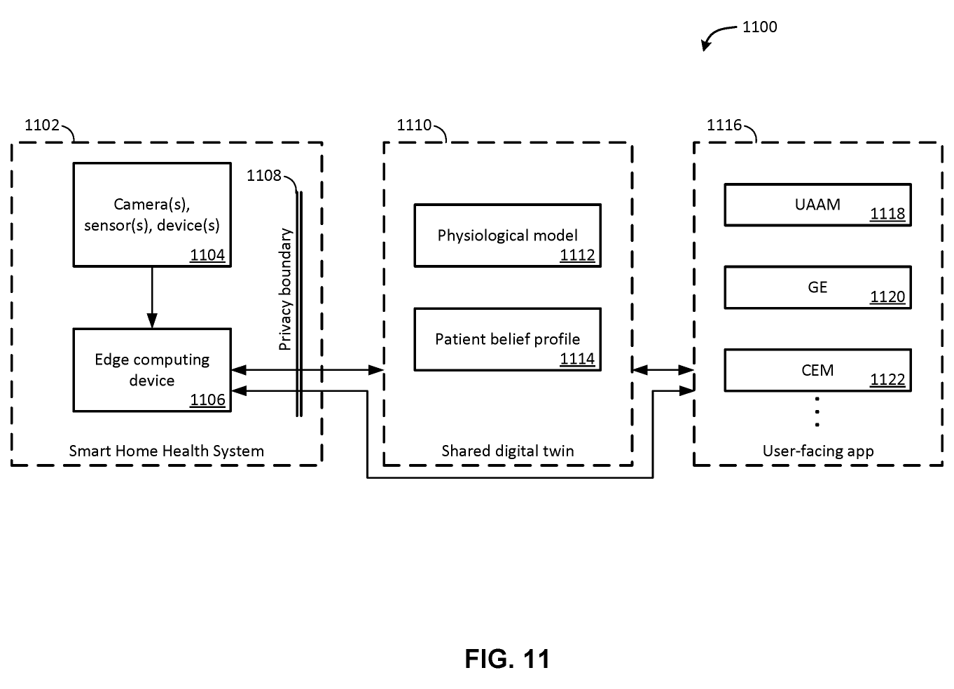

FIG. 11 illustrates an integration architecture 1100 for combining the home-based remote monitoring subsystem with the user-facing application via a shared digital twin according to some embodiments. The integration architecture 1100 may be provided as an example, and embodiments may vary in the specific components, data flows, or integration mechanisms involved. The integration architecture 1100 may represent how the home-based remote monitoring subsystem 110 and the user-facing application 102 shown in FIG. 1 interact through the shared digital twin database 108.

The integration architecture 1100 may comprise a smart home health system 1102, a shared digital twin 1110, and a user-facing application 1116. The smart home health system 1102 may be utilized within a home environment and/or other space occupied by an individual, such that cameras, sensors, and devices 1104 are deployed to monitor the individual. The cameras, sensors, and devices 1104 may include the RGB cameras 904, thermal cameras 906, smart mirrors 908, smart scales 910, and other devices and/or sensors 912 shown in FIG. 9. An edge computing device 1106 may be positioned within the home environment to process data from the cameras, sensors, and devices 1104. The edge computing device 1106 may correspond to the edge computing device 914 shown in FIG. 9.

A privacy boundary 1108 may be indicated between the smart home health system 1102 and external systems. The privacy boundary may represent the edge of the home environment where raw image data is deleted and only derived outputs are transmitted. Raw image data captured by cameras of the smart home health system 1102 may be processed locally by the edge computing device 1106 and deleted before any data leaves the home environment, preserving the individual’s privacy.

A shared digital twin 1110 may correspond to the digital twin database 108 shown in FIG. 1. Inside the shared digital twin 1110, a physiological model 1112 may represent the hierarchical physiological model of the individual’s body systems, corresponding to the digital twin structure 300 shown in FIG. 3A. A patient belief profile 1114 may represent the individual’s health-related beliefs, corresponding to the patient belief profile 320 shown in FIG. 3B. The physiological model 1112 and the patient belief profile 1114 may be stored together as part of the unified digital twin for the individual.

The edge computing device 1106 may transmit derived outputs across the privacy boundary 1108 to the shared digital twin 1110. The derived outputs may include activity timelines, inferred physiological parameters, risk scores, and alert flags generated by the edge computing device internal processing architecture 1000 shown in FIG. 10. These derived outputs may be incorporated into the physiological model 1112, updating the individual’s digital twin with information from the home-based monitoring subsystem.

A user-facing application 1116 may correspond to the user-facing application 102 shown in FIG. 1 and may have the internal architecture shown in FIG. 2. Inside the user-facing application 1116, a user-facing app agent module (UAAM) 1118 may provide personalized coaching and conversational interaction with the individual. The UAAM 1118 may correspond to the user-facing app agent module 204 shown in FIG. 2. A gamification engine (GE) 1120 may implement reward mechanisms to encourage healthy behaviors. The GE 1120 may correspond to the gamification engine 212 shown in FIG. 2. A community engagement module (CEM) 1122 may enable social interaction among individuals using the application. The CEM 1122 may correspond to the community engagement module 214 shown in FIG. 2. Additional modules may be present in the user-facing application 1116, as indicated by the dotted line below the CEM 1122.

The user-facing application 1116 may communicate bidirectionally with the shared digital twin 1110, as indicated by a double-headed arrow between the two regions. The user-facing application 1116 may retrieve information from the physiological model 1112 and the patient belief profile 1114 to personalize coaching, educational content, and health interventions delivered to the individual. The user-facing application 1116 may also transmit data collected from the individual, such as food logs, user interactions, conversational data, and behavioral observations, back to the shared digital twin 1110 for incorporation into the physiological model 1112 and the patient belief profile 1114.

In some embodiments, when the smart home health system 1102 is present and includes the edge computing device 1106, the community engagement module (CEM) 1122 may interface with the edge computing device 1106 to enable community-based features that leverage home monitoring data. For example, the CEM 1122 may retrieve anonymized activity summaries or achievement milestones from the edge computing device 1106 to enable the individual to share wellness achievements with the community without transmitting raw image data. The CEM 1122 may also receive contextual triggers from the edge computing device 1106 to prompt community engagement at appropriate times, such as after a detected exercise session or meal preparation activity.

According to some embodiments, one feature of the integration architecture 1100 is the event-triggered cross-system interaction capability. A detected activity state in the activity timeline generated by the edge computing device 1106 may trigger delivery of a personalized health intervention to the individual by the user-facing application 1116 based on the shared digital twin 1110. For example, if the edge computing device 1106 detects that the individual has entered the kitchen, this activity state may be transmitted to the shared digital twin 1110 and may trigger the UAAM 1118 to deliver a contextual meal suggestion or nutritional guidance to the individual via the user-facing application 1116. Similarly, if the user-facing application 1116 detects that the individual has logged a meal, this information may be transmitted to the shared digital twin 1110 and may influence the baseline comparison and risk scoring performed by the edge computing device 1106.

Through the integration architecture 1100, the home-based remote monitoring subsystem and the user-facing application may work together as a unified system, sharing data via the shared digital twin 1110 while maintaining privacy through the privacy boundary 1108. This integrated approach may enable more comprehensive, context-aware, and effective personalized health management than either subsystem could provide independently.

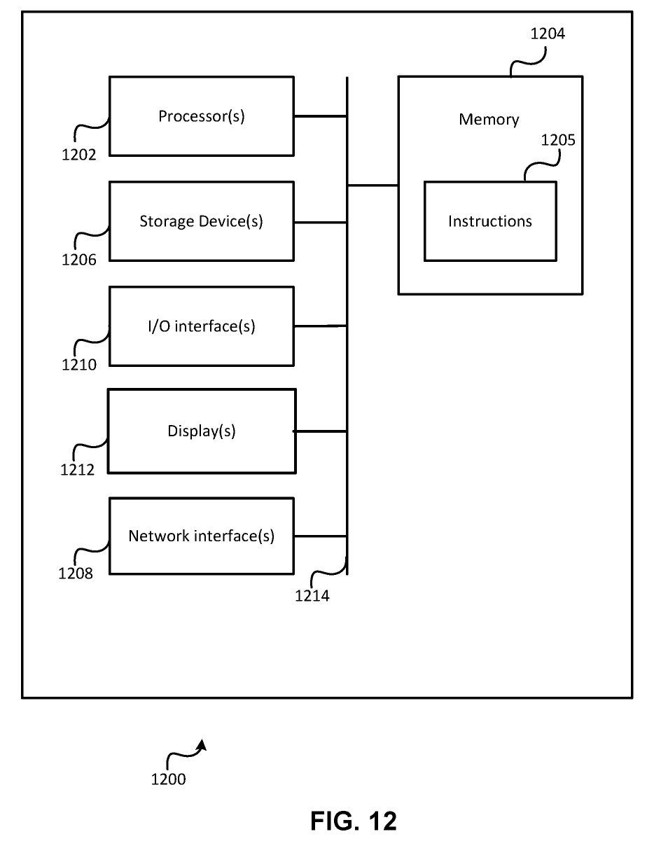

FIG. 12 illustrates a computer system 1200 according to some embodiments. The computer system 1200 may be provided as an example of hardware that can be used to implement various components described herein, such as the edge computing device 914 shown in FIG. 9, the human health processing system 104 shown in FIG. 1, the human health knowledge model server 106 shown in FIG. 1, the digital twin database 108 shown in FIG. 1, or other computing components of the system architecture 100. The computer system 1200 may vary in configuration and capabilities depending on the specific implementation.

The computer system 1200 may include one or more processors 1202, which may comprise one or more central processing units (CPUs), graphics processing units (GPUs), application-specific integrated circuits (ASICs), field-programmable gate arrays (FPGAs), or other processing units. The one or more processors 1202 may execute instructions stored in memory to perform the various processing operations described herein, such as the internal processing architecture 1000 shown in FIG. 10, the knowledge comparison operations described in FIG. 4, the digital twin simulation operations described in FIG. 7B, or other computational tasks.

The computer system 1200 may include one or more memory devices 1204, which may comprise random access memory (RAM), read-only memory (ROM), flash memory, or other volatile or non-volatile memory technologies. The one or more memory devices 1204 may store instructions, data, models, and other information used by the one or more processors 1202. For example, the one or more memory devices 1204 may store machine learning models used for pose estimation and activity classification, physiological parameter inference, or other AI-based processing operations. The one or more memory devices 1204 may also store digital twin data, patient belief profiles, activity timelines, or other application data.

The computer system 1200 may include one or more storage devices 1206, which may comprise hard disk drives, solid-state drives, network-attached storage, or other persistent storage technologies. The one or more storage devices 1206 may store larger datasets, historical data, databases, or other information that persists across system restarts. For example, the one or more storage devices 1206 may store the digital twin database 108, the human health knowledge model, historical activity timelines, or population-level aggregated data.

The computer system 1200 may include one or more network interfaces 1208, which may enable communication with other systems via a network. The one or more network interfaces 1208 may support wired or wireless communication protocols such as Ethernet, Wi-Fi, cellular networks, Bluetooth, or other networking technologies. The one or more network interfaces 1208 may enable the computer system 1200 to communicate with the network 920 shown in FIG. 9, transmit derived outputs from the edge computing device to the digital twin database, receive video feeds from cameras, or exchange data with other components of the system architecture 100.

The computer system 1200 may include one or more input/output (I/O) devices 1210, which may enable connection to various peripheral devices and user input mechanisms. The one or more I/O devices 1210 may include USB ports, video capture interfaces, display outputs, audio interfaces, keyboards, mice, touchscreens, microphones, cameras, or other input and output connection types. For example, when the computer system 1200 is used to implement the edge computing device 914, the one or more I/O devices 1210 may include camera input interfaces 1002 shown in FIG. 10 for receiving video feeds from RGB cameras and thermal cameras. The one or more I/O devices 1210 may enable user interactions with the user-facing application 102, clinician interface 114, or other components of the system.

The computer system 1200 may include one or more displays 1212, which may present visual information to an individual. The one or more displays 1212 may comprise a monitor, touchscreen, or other display technology. When the computer system 1200 is used to implement a user-facing application 102 on a mobile device or tablet, the one or more displays 1212 may present the user interface module 202 shown in FIG. 2. When the computer system 1200 is used to implement the clinician interface 114, the one or more displays 1212 may present the pre-visit AI briefing 806, digital twin summary view 810, or other clinician-facing information shown in FIG. 8.

The components of the computer system 1200 may be interconnected via one or more buses 1214 or other communication pathways. The one or more buses 1214 may enable data transfer between the one or more processors 1202, the one or more memory devices 1204, the one or more storage devices 1206, the one or more network interfaces 1208, the one or more I/O devices 1210, and the one or more displays 1212.

The computer system 1200 represents one example hardware configuration, and embodiments may be implemented using different hardware architectures, component selections, or system configurations. For example, the edge computing device 914 may be implemented as a dedicated embedded system with specialized hardware for video processing, while the human health processing system 104 may be implemented as a distributed cloud-based system comprising multiple interconnected servers. The specific hardware configuration may be selected based on performance requirements, cost constraints, power consumption considerations, or other design factors.

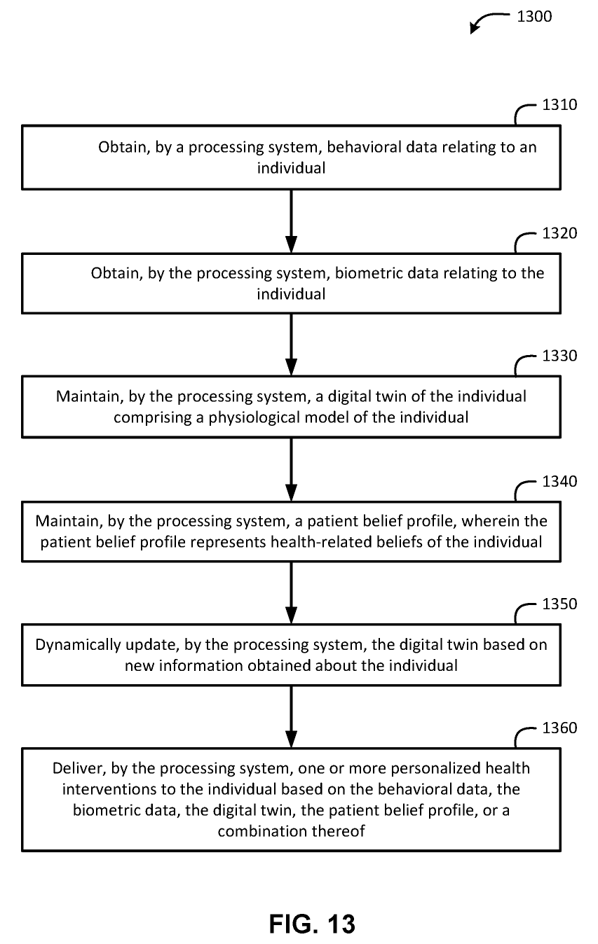

FIG. 13 illustrates a method 1300 for personalized health management according to some embodiments. The method 1300 may be provided as an example, and embodiments may vary in the specific operations, sequence, or implementation details involved. The method 1300 may represent operations performed by the human health processing system 104 shown in FIG. 1, in coordination with the user-facing application 102, the digital twin database 108, and other components of the system architecture 100.

At operation 1310, the method may include obtaining behavioral data relating to an individual. The behavioral data may be received by a processing system from various sources. The behavioral data may include observed behaviors such as meal choices, exercise patterns, sleep habits, medication adherence, social interactions, community engagement, responses to prompts, selections made within the user-facing application 102, and other actions performed by the individual. The behavioral data may be collected by the data acquisition module 206 shown in FIG. 2, which may interface with sensors on the device executing the application, such as accelerometers, gyroscopes, or GPS receivers. The behavioral data may also be collected from external devices and APIs 112 shown in FIG. 1, such as wearable devices, fitness trackers, or third-party health applications. In some embodiments, the behavioral data may include activity timelines generated by the home-based remote monitoring subsystem 110 shown in FIG. 1. The edge computing device 914 shown in FIG. 9 may process image data locally to generate activity classifications such as sleeping, walking, cooking, or eating, as described in the internal processing architecture 1000 shown in FIG. 10. These activity classifications may be transmitted as derived outputs to the processing system and may constitute behavioral data relating to the individual.

At operation 1320, the method may include obtaining biometric data relating to the individual. The biometric data may be received by the processing system from various sources. The biometric data may include physiological measurements such as heart rate, blood pressure, blood glucose levels, ketone levels, body weight, body composition, body temperature, respiratory rate, oxygen saturation, or other health-related metrics. The biometric data may be collected from wearable devices such as continuous glucose monitors, heart rate monitors, smart scales 910 shown in FIG. 9, or other biosensors. The biometric data may also be collected from cameras or sensors for capturing biometric measurements, including external device(s)/API(s) 112 of FIG. 1, as noted in the embodiments described herein. In some embodiments, the biometric data may include inferred physiological parameters generated by the home-based remote monitoring subsystem 110. The edge computing device 914 may apply machine learning models to infer physiological parameters such as heart rate, respiratory rate, body mass index, body fat percentage, or glycemic risk from image data, as described in the physiological parameter inference operations at block 1008 of FIG. 10. The biometric data may also include thermal data processed by the thermal processing module at block 1010 of FIG. 10 to estimate skin temperature and correlate thermal patterns with physiological states.

At operation 1330, the method may include maintaining a digital twin of the individual, comprising a physiological model of the individual. The digital twin may be maintained by the processing system and may be stored in the digital twin database 108 shown in FIG. 1. The digital twin may comprise a hierarchical physiological model that decomposes the human body into a plurality of interconnected systems, as shown in the digital twin structure 300 of FIG. 3A. The physiological model may include a circulatory and respiratory system 304, a digestive and excretory system 306, an immune and integumentary system 308, a nervous and endocrine system 310, and a musculoskeletal system 312. Each system may include associated parameters that are dynamically updated based on incoming behavioral data, biometric data, sensor measurements, and clinical observations. The physiological systems may be modeled using one or more types of models, such as mathematical models, machine learning models, artificial intelligence models, statistical models, or hybrid models combining multiple modeling approaches. The digital twin may be calibrated or personalized over time as new data becomes available, thereby improving the model’s accuracy for the individual. A local copy of the digital twin may be maintained by the digital twin module 208 shown in FIG. 2 for offline access and rapid retrieval, and may be synchronized with the remote digital twin database 108 via the cloud digital twin interface module 210.

At operation 1340, the method may include maintaining a patient belief profile associated with the digital twin, wherein the patient belief profile represents health-related beliefs of the individual. The patient belief profile may be maintained by the processing system and may be stored in association with the digital twin in the digital twin database 108. The patient belief profile may be constructed from multiple types of input data, as shown in the patient belief profile structure 320 of FIG. 3B. User interactions 322 may include responses to prompts, selections made within the user-facing application 102, and engagement with educational content. Behavioral data 324 may include observed behaviors such as meal choices, exercise patterns, and medication adherence that may reflect underlying beliefs about health. Biometric data 326 may include physiological measurements that may correlate with health-related beliefs or practices. Conversational data 328 may include text, audio, or video exchanges between the individual and the user-facing app agent module 204, from which statements about health beliefs may be extracted. The patient belief profile may be represented in a structured format, such as a structured database, a knowledge graph, a collection of belief statements with associated metadata, or as a machine learning model trained on the individual’s language and behavior patterns. The patient belief profile may be updated continuously or periodically as new input data becomes available through interactions with the user-facing application 102.

At operation 1350, the method may include dynamically updating the digital twin based on new information obtained about the individual. The processing system may update the digital twin as new behavioral data and biometric data are received. The dynamic updating may include updating physiological parameters in the hierarchical physiological model based on sensor measurements, updating activity timelines based on detected behaviors, updating inferred physiological parameters based on machine learning model outputs, and updating the patient belief profile based on new interactions with the individual. The digital twin module 208 shown in FIG. 2 may update the local digital twin based on newly collected data, and the cloud digital twin interface module 210 may synchronize the local digital twin with the remote digital twin database 108 when network connectivity is available, as described in the gamification state machine 500 shown in FIG. 5. The updating cloud digital twin state 522 may represent synchronization of the local digital twin with the remote digital twin database when network connectivity becomes available. The dynamic updating may enable the digital twin to continuously reflect the individual’s current physiological state, behavioral patterns, and health-related beliefs.

At operation 1360, the method may include delivering one or more personalized health interventions to the individual based on the behavioral data, the biometric data, the digital twin, the patient belief profile, or a combination thereof. The processing system may deliver personalized health interventions via the user-facing application 102. The personalized health interventions may be generated based on the behavioral data obtained at operation 1310, the biometric data obtained at operation 1320, the current state of the digital twin maintained at operation 1330, the patient belief profile maintained at operation 1340, or any combination of these data sources. The personalized health interventions may also be generated based on detected activity patterns, identified knowledge gaps or false beliefs, and other information maintained in the digital twin. In some embodiments, the personalized health interventions may be generated without clinician involvement, such that the individual may use the system independently based on the behavioral data, the biometric data, the digital twin, and the patient belief profile. In some embodiments, the personalized health interventions may additionally or alternatively be generated based on clinician input received, for example, via the clinician interface 114 shown in FIG. 1. The clinician input may include lifestyle prescriptions, behavioral mandates, recommendations, goals, or instructions provided by a healthcare provider for the individual. The clinician input may relate to daily behaviors such as physical activity targets, dietary targets, sleep hygiene practices, stress management activities, or other lifestyle modifications. The clinician input may additionally or alternatively include medication prescriptions specifying prescribed medications, dosages, administration schedules, or other pharmacological directives for the individual. The processing system may incorporate both lifestyle prescriptions and medication prescriptions into the personalized health interventions. For example, a clinician may prescribe a target amount of daily physical activity, such as walking a specified distance per day, or a dietary target, such as consuming a specified quantity of vegetables (or grams of fiber) per day. In another example, a clinician may prescribe a medication to be taken at a specified time each day, and the processing system may generate medication reminders, track adherence based on user input or sensor data, and deliver educational content about the prescribed medication. The processing system may receive the clinician input and incorporate the clinician input into the generation of personalized health interventions for the individual. The clinician-to-patient coaching recommendations module 812 shown in FIG. 8 may transmit the clinician input to the human health processing system 104 for incorporation into the personalized health interventions. The user-facing application 102 may present the clinician-prescribed goals or mandates in a dedicated area of the user interface module 202, such that the individual may view clinician-prescribed targets alongside system-generated recommendations. The user-facing app agent module 204 may incorporate the clinician-prescribed goals into messaging delivered via conversational interactions to the individual, such as reminders, encouragement, progress updates, or contextual prompts related to the clinician-prescribed targets. The gamification engine 212 shown in FIG. 2 may incorporate the clinician-prescribed goals into reward criteria, such that the individual may receive rewards for achieving or making progress toward clinician-prescribed targets. For example, if a clinician has prescribed two miles of walking per day, the user-facing app agent module 204 may deliver a message to the individual indicating progress toward the daily walking target, suggest a time or route for a walk based on the individual's current activity timeline, or provide encouragement upon completion of the prescribed activity, and the gamification engine 212 may award points or other rewards upon completion of the prescribed walking distance. The personalized health interventions may include educational content, predicted physiological outcomes based on simulation of the digital twin, personalized audio outputs such as guided meditation, meal plans, rewards for healthy behaviors, and other interventions tailored to the individual’s current understanding and health state. The user-facing app agent module 204 shown in FIG. 2 may deliver the personalized health interventions via the user interface module 202. The message flow diagram 600 shown in FIG. 6 illustrates how the user-facing app agent module may query the digital twin module for current physiological and belief profile states, transmit the belief profile state to the human health processing system, receive identified knowledge gaps or false beliefs from the human health processing system, and deliver personalized coaching and education to the user based on the identified gaps.

The method 1300 may further include additional operations in some embodiments. The method may include interfacing with a human health knowledge model comprising a curated knowledge base of human physiology and interactions with diet, exercise, pharmacological agents, and environmental factors, as shown in the human health knowledge model structure 400 of FIG. 4. The method may include comparing the patient belief profile against the human health knowledge model to identify one or more knowledge gaps or false beliefs of the individual, as shown in the knowledge comparison engine 404 of FIG. 4. The method may include delivering educational content to the individual based on an identified knowledge gap or false belief, as shown in the learning and training activity generator 408 of FIG. 4.

In some embodiments, the method may include simulating the digital twin based on one or more behaviors or actions recorded for the individual to generate a predicted physiological outcome, and outputting the predicted physiological outcome to the individual. The food logging and digital twin simulation workflow 720 shown in FIG. 7B illustrates this operation. At block 724, a digital twin may be simulated based on logged intake. At block 726, predicted blood glucose and ketone levels may be generated over time. At block 728, if the prediction indicates the user should not be hungry yet, the system may discourage further eating by outputting the predicted physiological outcome to the individual.

In some embodiments, the method may include providing a reward to an individual for performing one or more healthy behaviors, and providing a reward to each of two or more individuals based on performance of a healthy behavior by the two or more individuals. The gamification state machine 500 shown in FIG. 5 illustrates these operations. The reward allocation state 526 may calculate and award rewards to the individual based on detected healthy behaviors. The calculating rewards state 527 may calculate reward points or other incentives, and the celebrating user behavior state 528 may present the awarded rewards to the individual. The gamification engine 212 shown in FIG. 2 may support individual rewards and group rewards for behaviors performed by multiple individuals.

In some embodiments, the method may include generating a meal plan for the individual based on an image of food items available to the individual and simulating the digital twin based on the meal plan to predict a physiological response. The meal planning workflow 700 shown in FIG. 7A illustrates these operations. At block 702, the individual may capture images of refrigerator or cabinet contents. At block 704, the system may identify available food items from images. At block 706, a meal plan may be generated based on available items and digital twin parameters. The digital twin may be simulated based on the meal plan to predict physiological responses such as blood glucose levels, enabling the system to optimize the meal plan to support the user’s health goals.

In some embodiments, the method may include capturing, by one or more cameras of a home-based monitoring subsystem, image data of the individual within a home environment; processing, by an edge computing device of the home-based monitoring subsystem, the image data locally to generate an activity timeline comprising a time-series of activity states and inferred physiological parameters of the individual; transmitting derived outputs from the activity timeline for incorporation into the digital twin; and deleting raw image data on the edge computing device after processing such that no raw image data is transmitted outside the home environment. The home-based remote monitoring system architecture 900 shown in FIG. 9 illustrates these operations. The RGB cameras 904 and thermal cameras 906 may capture image data of the individual within the home environment 902. The edge computing device 914 may process the image data locally using the internal processing architecture 1000 shown in FIG. 10. The timeline generation module at block 1012 may generate an activity timeline comprising a time-series of activity states and inferred physiological parameters. The privacy management module at block 1020 may delete raw image data after processing to ensure that no raw image data is transmitted outside the home environment 902 via the network 920. The derived outputs may be transmitted to the digital twin database 928 for incorporation into the digital twin.

Through the method 1300, the system may provide continuous, personalized health management that integrates behavioral data, biometric data, digital twin modeling, patient belief profiling, and targeted interventions to support improved health outcomes for individuals.

In some embodiments, the system may leverage awareness of potentially undesirable physiological outcomes as a motivational element to encourage behavior change. When the digital twin simulation predicts an outcome that may be concerning to the individual, such as elevated blood glucose levels, increased cardiovascular risk, or other adverse physiological states, the system may present this information in a supportive, educational manner that motivates the individual to adopt healthier behaviors. The presentation may emphasize the individual’s agency and ability to avoid the undesirable outcome through specific actions, rather than using fear in a negative or demeaning way. For example, if the simulation predicts that continuing a current dietary pattern may lead to poor glycemic control, the system may present this prediction alongside actionable alternatives that would lead to more favorable outcomes. The system may frame the communication to empower the individual with knowledge about potential consequences while providing clear pathways to better health outcomes. This approach may be more effective than positive reinforcement alone for some individuals, as awareness of potential adverse consequences can serve as a powerful motivator for behavior change.

In some embodiments, when outputting the predicted physiological outcome to the individual, the method may include presenting information about potentially undesirable or concerning outcomes in a manner designed to motivate behavior change. The presentation may emphasize the individual’s ability to avoid the undesirable outcome through specific actions, providing both awareness of potential consequences and clear pathways to better outcomes. For example, if the digital twin simulation predicts that a proposed behavior may lead to adverse physiological consequences, the system may present this prediction alongside alternative behaviors that would lead to more favorable outcomes. The method may calibrate the emphasis on potential adverse outcomes based on the individual’s psychological profile, health literacy, and observed responsiveness to different motivational approaches. For some individuals, awareness of what they are working to avoid may be as motivating or more motivating than awareness of what they are working to achieve. The method may balance risk-awareness messaging with supportive guidance and positive reinforcement to create a comprehensive motivational framework that empowers the individual to make informed health decisions.

In view of this description, embodiments may include different combinations of features. Implementation examples are described in the following numbered clauses:

Clause 1: A system comprising one or more processors; and one or more processor-readable media storing instructions which, when executed by the one or more processors, cause performance of operations comprising obtaining behavioral data relating to an individual; obtaining biometric data relating to the individual; maintaining a digital twin of the individual comprising a physiological model of the individual; maintaining a patient belief profile, wherein the patient belief profile represents health-related beliefs of the individual; dynamically updating the digital twin based on new information obtained about the individual; and delivering one or more personalized health interventions to the individual based on the behavioral data, the biometric data, the digital twin, the patient belief profile, or a combination thereof.

Clause 2: The system of clause 1, wherein the operations further comprise interfacing with a human health knowledge model comprising a curated knowledge base of human physiology; comparing the patient belief profile against the human health knowledge model to identify a knowledge gap or false belief of the individual; and providing information to the individual based on the identified knowledge gap or false belief.

Clause 3: The system of any one of clauses 1-2, wherein the physiological model comprises a plurality of parameters corresponding to components of the human body.

Clause 4: The system of any one of clauses 1-3, wherein the operations further comprise simulating the digital twin based on one or more behaviors or actions of the individual to generate a predicted physiological outcome; and presenting the predicted physiological outcome.

Clause 5: The system of any one of clauses 1-4, wherein the operations further comprise providing a reward to the individual for performing one or more healthy behaviors.

Clause 6: The system of clause 5, wherein the operations further comprise providing a reward to each of one or more additional individuals based on performing the one or more healthy behaviors with the individual.

Clause 7: The system of any one of clauses 1-6, wherein the edge computing device or an application executed by a user device is configured to: receive audio sounds via microphones; and send audio sounds via speakers.

Clause 8: The system of any one of clauses 1-7, wherein the operations further comprise generating a meal plan for the individual based on an image of food items available to the individual; and simulating the digital twin based on the meal plan to predict a physiological response.

Clause 9: The system of any one of clauses 1-8, wherein the operations further comprise communicating one or more aspects of the digital twin to a clinical user via a clinician interface.

Clause 10: The system of any one of clauses 1-9, further comprising a home-based monitoring subsystem comprising an edge computing device configured to obtain image data from one or more cameras of the individual within a home environment; process the image data locally on the edge computing device to generate an activity timeline comprising a time-series of activity states and inferred physiological parameters of the individual; and transmit derived outputs from the activity timeline for incorporation into the digital twin.

Clause 11: The system of clause 10, wherein the edge computing device is configured to delete raw image data after processing such that no raw image data is transmitted outside the home environment.

Clause 12: The system of any one of clauses 10-11, wherein the edge computing device is configured to apply a first machine learning model to the image data to detect a pose and classify an activity of the individual, and apply a second machine learning model to an output of the first machine learning model to infer one or more physiological parameters of the individual.

Clause 13: The system of any one of clauses 10-12, wherein the edge computing device is further configured to compare the activity timeline against a personalized baseline of the individual to detect an anomaly; generate a risk score based on the detected anomaly; and transmit an alert based on the risk score exceeding a threshold.

Clause 14: A method comprising obtaining, by a processing system, behavioral data relating to an individual; obtaining, by the processing system, biometric data relating to the individual; maintaining, by the processing system, a digital twin of the individual comprising a physiological model of the individual; maintaining, by the processing system, a patient belief profile, wherein the patient belief profile represents health-related beliefs of the individual; dynamically updating, by the processing system, the digital twin based on new information obtained about the individual; and delivering, by the processing system, one or more personalized health interventions to the individual based on the behavioral data, the biometric data, the digital twin, the patient belief profile, or a combination thereof.

Clause 15: The method of clause 14, further comprising interfacing with a human health knowledge model comprising a curated knowledge base of human physiology; comparing the patient belief profile against the human health knowledge model to identify a knowledge gap or false belief of the individual; and providing information to the individual based on the identified knowledge gap or false belief.

Clause 16: The method of any one of clauses 14-15, further comprising simulating the digital twin based on one or more behaviors or actions recorded for the individual to generate a predicted physiological outcome; and presenting the predicted physiological outcome.

Clause 17: The method of any one of clauses 14-16, further comprising providing a reward to the individual for performing one or more healthy behaviors.

Clause 18: The method of any one of clauses 14-17, further comprising generating a meal plan for the individual based on an image of food items available to the individual and simulating the digital twin based on the meal plan to predict a physiological response.

Clause 19: The method of any one of clauses 14-18, further comprising obtaining, from one or more cameras of a home-based monitoring subsystem, image data of the individual within a home environment; processing, by an edge computing device of the home-based monitoring subsystem, the image data locally to generate an activity timeline comprising a time-series of activity states and inferred physiological parameters of the individual; transmitting derived outputs from the activity timeline to the processing system for incorporation into the digital twin; and deleting raw image data on the edge computing device after processing such that no raw image data is transmitted outside the home environment.

Clause 20: A non-transitory computer-readable medium storing instructions that, when executed by a processing system, cause the processing system to obtain behavioral data relating to an individual; obtain biometric data relating to the individual; maintain a digital twin of the individual comprising a physiological model of the individual; maintain a patient belief profile, wherein the patient belief profile represents health-related beliefs of the individual; dynamically update the digital twin based on new information obtained about the individual; and deliver one or more personalized health interventions to the individual based on the behavioral data, the biometric data, the digital twin, the patient belief profile, or a combination thereof.

Clause 21: A system comprising one or more cameras configured to capture image data of an individual within a home environment; an edge computing device configured to process the image data locally to generate an activity timeline comprising a time-series of activity states and inferred physiological parameters of the individual, apply a first machine learning model to the image data to detect a pose and classify an activity of the individual, apply a second machine learning model to an output of the first machine learning model to infer one or more physiological parameters of the individual, compare the activity timeline against a personalized baseline of the individual to detect an anomaly and generate a risk score based on the detected anomaly, and transmit an alert to the individual, a caregiver, or a healthcare provider based on the risk score exceeding a threshold; and a processing system configured to receive derived outputs from the activity timeline and incorporate the derived outputs into a digital twin of the individual.

What is claimed is:

1. A system comprising:
one or more processors; and
one or more processor-readable media storing instructions which, when executed by the one or more processors, cause performance of operations comprising:
obtaining behavioral data relating to an individual;
obtaining biometric data relating to the individual;
maintaining a digital twin of the individual comprising a physiological model of the individual;
maintaining a patient belief profile, wherein the patient belief profile represents health-related beliefs of the individual;
dynamically updating the digital twin based on new information obtained about the individual; and
delivering one or more personalized health interventions to the individual based on the behavioral data, the biometric data, the digital twin, the patient belief profile, or a combination thereof.

2. The system of claim 1, wherein the operations further comprise:
interfacing with a human health knowledge model comprising a curated knowledge base of human physiology;
comparing the patient belief profile against the human health knowledge model to identify a knowledge gap or false belief of the individual; and
providing information to the individual based on the identified knowledge gap or false belief.

3. The system of claim 1, wherein the physiological model comprises a plurality of parameters corresponding to components of the human body.

4. The system of claim 1, wherein the operations further comprise:
simulating the digital twin based on one or more behaviors or actions of the individual to generate a predicted physiological outcome; and
presenting the predicted physiological outcome.

5. The system of claim 1, wherein the operations further comprise providing a reward to the individual for performing one or more healthy behaviors.

6. The system of claim 5, wherein the operations further comprise providing a reward to each of one or more additional individuals based on performing the one or more healthy behaviors with the individual.

7. The system of claim 1, wherein the edge computing device or an application executed by a user device is configured to:
receive audio sounds via microphones; and
send audio sounds via speakers.

8. The system of claim 1, wherein the operations further comprise:
generating a meal plan for the individual based on an image of food items available to the individual; and
simulating the digital twin based on the meal plan to predict a physiological response.

9. The system of claim 1, wherein the operations further comprise communicating one or more aspects of the digital twin to a clinical user via a clinician interface.

10. The system of claim 1, further comprising a home-based monitoring subsystem comprising an edge computing device configured to:
obtain image data from one or more cameras of the individual within a home environment;
process the image data locally on the edge computing device to generate an activity timeline comprising a time-series of activity states and inferred physiological parameters of the individual; and
transmit derived outputs from the activity timeline for incorporation into the digital twin.

11. The system of claim 10, wherein the edge computing device is configured to delete raw image data after processing such that no raw image data is transmitted outside the home environment.

12. The system of claim 10, wherein the edge computing device is configured to:
apply a first machine learning model to the image data to detect a pose and classify an activity of the individual, and
apply a second machine learning model to an output of the first machine learning model to infer one or more physiological parameters of the individual.

13. The system of claim 10, wherein the edge computing device is further configured to:
compare the activity timeline against a personalized baseline of the individual to detect an anomaly;
generate a risk score based on the detected anomaly; and
transmit an alert based on the risk score exceeding a threshold.

14. A method comprising:
obtaining, by a processing system, behavioral data relating to an individual;
obtaining, by the processing system, biometric data relating to the individual;
maintaining, by the processing system, a digital twin of the individual comprising a physiological model of the individual;
maintaining, by the processing system, a patient belief profile, wherein the patient belief profile represents health-related beliefs of the individual;
dynamically updating, by the processing system, the digital twin based on new information obtained about the individual; and
delivering, by the processing system, one or more personalized health interventions to the individual based on the behavioral data, the biometric data, the digital twin, the patient belief profile, or a combination thereof.

15. The method of claim 14, further comprising:
receiving, by the processing system, clinician input from a healthcare provider via a clinician interface, the clinician input comprising one or more of a lifestyle prescription specifying target behaviors for the individual or a medication prescription specifying a prescribed medication for the individual; and
incorporating, by the processing system, the clinician input into the one or more personalized health interventions delivered to the individual.

16. The method of claim 14, further comprising:
simulating the digital twin based on one or more behaviors or actions recorded for the individual to generate a predicted physiological outcome; and
presenting the predicted physiological outcome.

17. The method of claim 14, further comprising providing a reward to the individual for performing one or more healthy behaviors.

18. The method of claim 14, further comprising generating a meal plan for the individual based on an image of food items available to the individual and simulating the digital twin based on the meal plan to predict a physiological response.

19. The method of claim 14, further comprising:
obtaining, from one or more cameras of a home-based monitoring subsystem, image data of the individual within a home environment;
processing, by an edge computing device of the home-based monitoring subsystem, the image data locally to generate an activity timeline comprising a time-series of activity states and inferred physiological parameters of the individual;
transmitting derived outputs from the activity timeline to the processing system for incorporation into the digital twin; and
deleting raw image data on the edge computing device after processing such that no raw image data is transmitted outside the home environment.

20. A non-transitory computer-readable medium storing instructions that, when executed by a processing system, cause the processing system to:
obtain behavioral data relating to an individual;
obtain biometric data relating to the individual;
maintain a digital twin of the individual comprising a physiological model of the individual;
maintain a patient belief profile, wherein the patient belief profile represents health-related beliefs of the individual;
dynamically update the digital twin based on new information obtained about the individual; and
deliver one or more personalized health interventions to the individual based on the behavioral data, the biometric data, the digital twin, the patient belief profile, or a combination thereof.
